(19) United States
(12) Patent Application Publication
(10) Pub. No.: US 2025/0001173 A1
(43) Pub. Date: Jan. 2, 2025

(54) NEUROSTIMULATION CLOSED-LOOP PROGRAMMING RECOMMENDATION SYSTEM

(71) Applicant: Boston Scientific Neuromodulation Corporation, Valencia, CA (US)

(72) Inventors: Matthew Lee McDonald, Glendale, CA (US); Bradley Lawrence Hershey, Carrollton, TX (US); Amarpreet Singh Bains, Woodbury, MN (US); Dat Thanh Huynh, North Hollywood, CA (US); Marcus Harvey, San Francisco, CA (US)

(21) Appl. No.: 18/753,831

(22) Filed: Jun. 25, 2024

Related U.S. Application Data
(60) Provisional application No. 63/523,539, filed on Jun. 27, 2023.

(51) Int. Cl. A61N 1/36; A61N 1/372; G16H 20/40

(52) U.S. Cl. CPC A61N 1/3603; A61N 1/37247; G16H 20/40

(57) ABSTRACT

Systems and methods for generating and implementing customized recommendations as part of closed-loop neurostimulation programming and device configuration approaches are disclosed. In an example, a system is configured to: identify, with a recommendation model (e.g., a closed-loop programming algorithm), a programming setting for use in a neurostimulation device that controls neurostimulation treatment of a patient; communicate, to a patient device (e.g., a patient smartphone), a command to present a recommendation to use the programming setting; and communicate, to the patient device, data values associated with the programming setting, to reconfigure the neurostimulation device according to the programming setting. In various examples, a medical user interface enables a medical user (e.g., clinician) to customize the recommendation, and a patient user interface enables a patient to view and use the recommendation.

### CLAIM OF PRIORITY

This application claims the benefit of U.S. Provisional Application No. 63/523,539 filed on Jun. 27, 2023, which is hereby incorporated by reference in its entirety.

### TECHNICAL FIELD

This document relates generally to data processing obtained in connection with the use of medical devices, and more particularly, to systems, devices, and methods for generating and presenting recommendations in connection with implanted electrical stimulation, including human-guided programming recommendations for neurostimulation treatments used for pain treatment, movement disorders, and/or management of such conditions.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). A neurostimulation system can be used to electrically stimulate tissue or nerve centers to treat nervous or muscular disorders. For example, an SCS system may be configured to deliver electrical pulses to a specified region of a patient's spinal cord, such as particular spinal nerve roots or nerve bundles, to produce an analgesic effect that masks pain sensation, or to produce a functional effect that allows increased movement or activity of the patient. Other forms of neurostimulation may include a DBS system which uses similar pulses of electricity at particular locations in the brain to reduce symptoms of essential tremors, Parkinson's disease, psychological disorders, or the like.

Various approaches are being developed to enable personalized programming and optimized forms of programming used by neurostimulation systems, including partially or fully automated forms of generating or delivering specific neurostimulation parameters known as closed-loop programming. Some closed-loop programming approaches can, for example, customize changes to stimulator programs, or may suggest or automatically select a program when a particular condition is identified. However, due to changing patient needs and the many types of changes that can be implemented in neurostimulation systems, some patients (or clinicians/medical professionals who supervise such patients) may not be able to use closed-loop programming for their neurostimulation systems.

### SUMMARY

Example 1 is a system to generate recommendations for neurostimulation device configuration, the system comprising: one or more processors; and one or more memory devices comprising instructions, which when executed by the one or more processors, cause the one or more processors to: identify, with a recommendation model, a programming setting for use a neurostimulation device to control neurostimulation treatment of a patient; communicate, to a patient device, a command to present a recommendation to use the programming setting, wherein the recommendation is customized by another user, and wherein the patient device includes a patient user interface to present the recommendation to the patient; and communicate, to the patient device, data values associated with the programming setting, wherein the data values are used to reconfigure the neurostimulation device according to the programming setting.

In Example 2, the subject matter of Example 1 optionally includes subject matter where the another user is a medical user associated with care of the patient, and wherein the instructions further cause the one or more processors to: communicate, to a medical user device, the programming setting identified with the recommendation model; and receive, from the medical user device, a command to activate the recommendation for use of the programming setting, wherein the medical user device includes a medical user interface to enable the medical user to customize and activate the recommendation; wherein operations to communicate the command and the data values to the patient device occur after customization and activation of the recommendation by the medical user.

In Example 3, the subject matter of Example 2 optionally includes subject matter where the medical user interface presents the recommendation in a prioritized listing of multiple available recommendations for the patient, and wherein the medical user interface receives user input from the medical user to provide the customization and the activation of the recommendation.

In Example 4, the subject matter of any one or more of Examples 2-3 optionally include subject matter where the medical user interface receives user input from the medical user to modify at least a part of the programming setting or modify at least one presentation characteristic of the recommendation.

In Example 5, the subject matter of any one or more of Examples 1-4 optionally include subject matter where the patient user interface further includes encoded data values associated with the programming setting, and wherein the encoded data values are readable by a patient programming device to cause the patient programming device to reconfigure operation of the neurostimulation device according to the programming setting.

In Example 6, the subject matter of any one or more of Examples 1-5 optionally

include subject matter where the patient device includes programming functionality to directly communicate the programming setting to the neurostimulation device.

In Example 7, the subject matter of any one or more of Examples 1-6 optionally include subject matter where the patient device is a smartphone operable by the patient, and wherein the patient user interface is presented in a software app installed on the smartphone.

In Example 8, the subject matter of any one or more of Examples 1-7 optionally include subject matter where the recommendation to use the programming setting is associated with a recommended time of delivery or validity for the patient device.

In Example 9, the subject matter of any one or more of Examples 1-8 optionally include subject matter where the instructions further cause the one or more processors to: receive, from the patient device, feedback relating to the recommendation, wherein subsequent recommendations to reconfigure the neurostimulation device are customized to the patient based on the feedback.

In Example 10, the subject matter of Example 9 optionally includes subject matter where the recommendation model is further configured to evaluate data values based on the feedback relating to the recommendation, wherein the feedback relates to one or more of: sleep, pain, movement, emotional state, medication state, mobility, or activity, in connection with use of the neurostimulation treatment, and wherein the feedback is obtained from the patient using one or more questionnaires, surveys, freeform text inputs, voice inputs, or sensor data observations.

In Example 11, the subject matter of any one or more of Examples 1-10 optionally include subject matter where the recommendation model is further configured to evaluate device data collected during use of a program to perform the neurostimulation treatment.

In Example 12, the subject matter of any one or more of Examples 1-11 optionally include subject matter where the patient device is further configured to receive a user command to manually input or control the data values, to reconfigure a program in the neurostimulation device according to the programming setting.

In Example 13, the subject matter of any one or more of Examples 1-12 optionally include subject matter where the data values to reconfigure the neurostimulation device causes a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.

Example 14 is a machine-readable medium including instructions, which when executed by a machine, cause the machine to perform the operations of the system of any of the Examples 1 to 13.

Example 15 is a method to perform the operations of the system of any of the Examples 1 to 13.

Example 16 is a device to generate recommendations for neurostimulation device configuration, the device comprising: one or more processors; and one or more memory devices comprising instructions, which when executed by the one or more processors, cause the one or more processors to: identify, with a recommendation model, a programming setting for use a neurostimulation device to control neurostimulation treatment of a patient; communicate, to a patient device, a command to present a recommendation to use the programming setting, wherein the recommendation is customized by another user, and wherein the patient device includes a patient user interface to present the recommendation to the patient; and communicate, to the patient device, data values associated with the programming setting, wherein the data values are used to reconfigure the neurostimulation device according to the programming setting.

In Example 17, the subject matter of Example 16 optionally includes subject matter where the another user is a medical user associated with care of the patient, and wherein the instructions further cause the one or more processors to: communicate, to a medical user device, the programming setting identified with the recommendation model; and receive, from the medical user device, a command to activate the recommendation for use of the programming setting, wherein the medical user device includes a medical user interface to enable the medical user to customize and activate the recommendation; wherein operations to communicate the command and the data values to the patient device occur after customization and activation of the recommendation by the medical user.

In Example 18, the subject matter of any one or more of Examples 16-17 optionally include subject matter where the patient user interface further includes encoded data values associated with the programming setting, and wherein the encoded data values are readable by a patient programming device to cause the patient programming device to reconfigure operation of the neurostimulation device according to the programming setting.

In Example 19, the subject matter of any one or more of Examples 16-18 optionally include subject matter where the patient device includes programming functionality to directly communicate the programming setting to the neurostimulation device.

In Example 20, the subject matter of any one or more of Examples 16-19 optionally include subject matter where the patient device is a smartphone operable by the patient, and wherein the patient user interface is presented in a software app installed on the smartphone.

In Example 21, the subject matter of any one or more of Examples 16-20 optionally include subject matter where the recommendation to use the programming setting is associated with a recommended time of delivery or validity for the patient device.

In Example 22, the subject matter of any one or more of Examples 16-21 optionally include subject matter where the instructions further cause the one or more processors to: receive, from the patient device, feedback relating to the recommendation, wherein subsequent recommendations to reconfigure the neurostimulation device are customized to the patient based on the feedback.

In Example 23, the subject matter of Example 22 optionally includes subject matter where the recommendation model is further configured to evaluate data values based on the feedback relating to the recommendation, wherein the feedback relates to one or more of: sleep, pain, movement, emotional state, medication state, mobility, or activity, in connection with use of the neurostimulation treatment, and wherein the feedback is obtained from the patient using one or more questionnaires, surveys, freeform text inputs, voice inputs, or sensor data observations.

In Example 24, the subject matter of any one or more of Examples 16-23 optionally include subject matter where the recommendation model is further configured to evaluate device data collected during use of a program to perform the neurostimulation treatment.

In Example 25, the subject matter of any one or more of Examples 16-24 optionally include subject matter where the patient device is further configured to receive a user command to manually input or control the data values, to reconfigure a program in the neurostimulation device according to the programming setting; wherein the data values to reconfigure the neurostimulation device causes a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.

Example 26 is a method for generating recommendations for neurostimulation device configuration, comprising: identifying, with a recommendation model, a programming setting for use a neurostimulation device to control neurostimulation treatment of a patient; communicating, to a patient device, a command to present a recommendation to use the programming setting, wherein the recommendation is customized by another user, and wherein the patient device includes a patient user interface to present the recommendation to the patient; and communicate, to the patient device, data values associated with the programming setting, wherein the data values are used to reconfigure the neurostimulation device according to the programming setting.

In Example 27, the subject matter of Example 26 optionally includes subject matter where the another user is a medical user associated with care of the patient, and wherein the method further comprises: communicating, to a medical user device, the programming setting identified with the recommendation model; and receiving, from the medical user device, a command to activate the recommendation for use of the programming setting, wherein the medical user device includes a medical user interface to enable the medical user to customize and activate the recommendation; wherein communicating the command and the data values to the patient device occurs after customization and activation of the recommendation by the medical user.

In Example 28, the subject matter of any one or more of Examples 26-27 optionally include subject matter where the patient user interface further includes encoded data values associated with the programming setting, and wherein the encoded data values are readable by a patient programming device to cause the patient programming device to reconfigure operation of the neurostimulation device according to the programming setting.

In Example 29, the subject matter of any one or more of Examples 26-28 optionally include subject matter where the patient device includes programming functionality to directly communicate the programming setting to the neurostimulation device.

In Example 30, the subject matter of any one or more of Examples 26-29 optionally include subject matter where the patient device is a smartphone operable by the patient, and wherein the patient user interface is presented in a software app installed on the smartphone.

In Example 31, the subject matter of any one or more of Examples 26-30 optionally include subject matter where the recommendation to use the programming setting is associated with a recommended time of delivery or validity for the patient device.

In Example 32, the subject matter of any one or more of Examples 26-31 optionally include receiving, from the patient device, feedback relating to the recommendation, wherein subsequent recommendations to reconfigure the neurostimulation device are customized to the patient based on the feedback.

In Example 33, the subject matter of Example 32 optionally includes subject matter where the recommendation model is further configured to evaluate data values based on the feedback relating to the recommendation, wherein the feedback relates to one or more of: sleep, pain, movement, emotional state, medication state, mobility, or activity, in connection with use of the neurostimulation treatment, and wherein the feedback is obtained from the patient using one or more questionnaires, surveys, freeform text inputs, voice inputs, or sensor data observations.

In Example 34, the subject matter of any one or more of Examples 26-33 optionally include subject matter where the recommendation model is further configured to evaluate device data collected during use of a program to perform the neurostimulation treatment.

In Example 35, the subject matter of any one or more of Examples 26-34 optionally include subject matter where the patient device is further configured to receive a user command to manually input or control the data values, to reconfigure a program in the neurostimulation device according to the programming setting; wherein the data values to reconfigure the neurostimulation device causes a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.

This Summary is an overview of some of the teachings of the present application and not intended to be an exclusive or exhaustive treatment of the present subject matter. Further details about the present subject matter are found in the detailed description and appended claims. Other aspects of the disclosure will be apparent to persons skilled in the art upon reading and understanding the following detailed description and viewing the drawings that form a part thereof, each of which are not to be taken in a limiting sense. The scope of the present disclosure is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates a data processing system to analyze data and generate recommendations in connection with the use of neurostimulation programs and closed-loop neurostimulation programming, according to an example.

FIG. 2 illustrates a neuromodulation system as an example of a medical device system, according to an example.

FIG. 3 further illustrates a neuromodulation system as an ambulatory medical device, such as implemented by a neurostimulation treatment, according to an example.

FIG. 4 illustrates a neuromodulation device connected to a programming device, according to an example.

FIG. 5 illustrates data interactions with a closed-loop programming data service, for operation of a neuromodulation device with closed-loop programming, according to an example.

FIG. 6 depicts a workflow for closed-loop programming of a neuromodulation system, according to an example.

FIG. 7A, FIG. 7B, FIG. 7C, FIG. 7D, and FIG. 7E depict user interfaces provided on a medical user device for control of closed-loop programming operations, according to an example.

FIG. 8A, FIG. 8B, FIG. 8C, FIG. 8D, FIG. 8E, and FIG. 8F depict user interfaces provided on a patient computing device for implementation of closed-loop programming recommendations, according to an example.

FIG. 9A, FIG. 9B, FIG. 9C, FIG. 9D depict additional user interfaces provided on a patient computing device for implementation of closed-loop programming recommendations, according to an example.

FIG. 10A, FIG. 10B, and FIG. 10C depict user interfaces provided on a programming device for implementation of closed-loop programming recommendations, according to an example.

FIG. 11 illustrates data flows for implementation of closed-loop programming recommendations, according to an example.

FIGS. 12A and 12B illustrate further workflows for patient implementation of closed-loop programming recommendations, according to an example.

FIG. 13 illustrates a data processing flow for implementing recommended neurostimulation treatments in a human patient, including in response to closed-loop programming recommendations, according to an example.

FIG. 14 illustrates a processing method implemented by a system or device to generate and deploy recommendations for neurostimulation programming in connection with use of a closed-loop programming model, according to an example.

FIG. 15 illustrates, by way of example, a block diagram of an embodiment of a system (e.g., a computing system) for coordinating programming recommendations in connection with use of a closed-loop programming model, according to an example.

FIG. 16 illustrates, by way of example, a block diagram of an embodiment of a system (e.g., a computing system) implementing neurostimulation programming circuitry to cause programming of an implantable electrical neurostimulation device, according to an example.

FIG. 17 is a block diagram illustrating a machine in the example form of a computer system, within which a set or sequence of instructions may be executed to cause the machine to perform any one of the methodologies discussed herein, according to an example.

### DETAILED DESCRIPTION

The following detailed description of the present subject matter refers to the accompanying drawings which show, by way of illustration, specific aspects and embodiments in which the present subject matter may be practiced. These embodiments are described in sufficient detail to enable those skilled in the art to practice the present subject matter. Other embodiments may be utilized and structural, logical, and electrical changes may be made without departing from the scope of the present subject matter. References to “an”, “one”, or “various” embodiments in this disclosure are not necessarily to the same embodiment, and such references contemplate more than one embodiment. The following detailed description is, therefore, not to be taken in a limiting sense, and the scope is defined only by the appended claims, along with the full scope of legal equivalents to which such claims are entitled.

Various embodiments of the present subject matter relate to user interfaces, data algorithms and models, data processing systems, workflows, and computing systems and devices used in connection with a closed-loop neurostimulation recommendation and programming system. As an example, aspects include how to generate, deliver, track, and implement messages relating to a closed-loop programming recommendation (e.g., recommended changes or settings for neurostimulation device programming) in a scenario where features of closed-loop programming are partially automated. Such recommendations may include patient-customized guidance or recommendations that effect changes to device programming schedules or settings (e.g., amplitude, duration, timing, frequency values). As another example, aspects refer to a patient recommendation framework to control the receipt, presentation, interaction, and use of data relating to a closed-loop programming recommendation. As yet another example, aspects refer to techniques for transferring recommendation data (including recommended programming settings) of a closed-loop programming recommendation between patient devices, such as to transfer program settings from a personal computing device (e.g., smartphone operating a recommendation app) to a neurostimulation control device (e.g., to a remote controller or implantable pulse generator that is not internet-connected).

Neurostimulation systems may provide a variety of programming options, and patients may have multiple needs or preferences that change over time. The following introduces digital assistant features to help patients track neurostimulation outcomes and receive assistance in a remote (home) setting from a clinician or a medical device support team. The following also enables patients to identify what programming options can be tried, and how the patient would benefit from education of how to adjust the neurostimulation system. The following also addresses dynamic options that are deployable in closed-loop recommendation systems, and explains how recommendations can be created and delivered via user interfaces and communications, efficiently transferred among systems, and presented with customized options that control the delivery and use of recommendations to patients.

Accordingly, one system implementation includes functionality that enables a medical professional user to choose, configure and create a customized recommendation (or, a system-generated customized recommendation) and choose to send the recommendation to a specific patient. Another system implementation includes functionality that enables a medical professional user to monitor and view the results of customized recommendations that were automatically sent to a specific patient. Another system implementation includes functionality that enables a user to view a customized recommendation and provide feedback (“likes”, freeform text, ratings) and the result of the recommendation (whether the recommendation action was used or disregarded). Finally, another system implementation uses data (stimulator data, patient input data, wearable data, etc.) and/or user input and/or feedback from previous recommendations to select and deliver recommendations. For instance, a particular recommendation may be selected from a predefined set of recommendations, and/or customized for a particular recipient, based on the medical user or patient systems discussed above.

The user interfaces discussed herein, for example, can output suggestions, recommendations, instructions, guidance, or other information to inform the human subject on what settings to change, what program to use, what therapies to try, and the like. Such user interface screens can also collect feedback from the user on whether a particular therapy is effective. Various forms of data processing may occur to generate these instructions, recommendations, and suggestions, and to process feedback or usage data produced from the use of the recommended therapy. Such features provide useful techniques to implement and improve a closed-loop programming system, including in settings where the recommendations are guided or approved by a clinician (e.g., doctor or medical professional responsible for the patient's care) or a third party (e.g., device manufacturer or service provider). This can lead to a variety of optimizations for new programs, modification of existing programs, improved program usage and schedules, and other neurostimulation device improvements.

The following describes a “data collection platform”, “data processing system”, and “data service” that generally refers to portions of a compute platform (e.g., a combination of hardware, firmware and software) with a set of capabilities for collecting, processing, and generating data in connection with closed-loop neurostimulation recommendations, programming, and the user interfaces discussed below. A compute platform may be a single platform (e.g., at a single cloud service) or may be organized as more than one platform (e.g., among multiple distributed computing locations) that are configured to cooperate with each other. A compute platform may obtain data from one device or from more than one device. Thus, a therapy device such as an implanted neuromodulation device may provide some portion of the collected data, and a user device (e.g., smartphone) with an interactive feedback user interface (e.g., provided by a smartphone app) may provide another portion of the collected data. A compute platform may also obtain data from other sensor(s) and other data source(s), and be guided by (or under the control of) a clinician or device manufacturer.

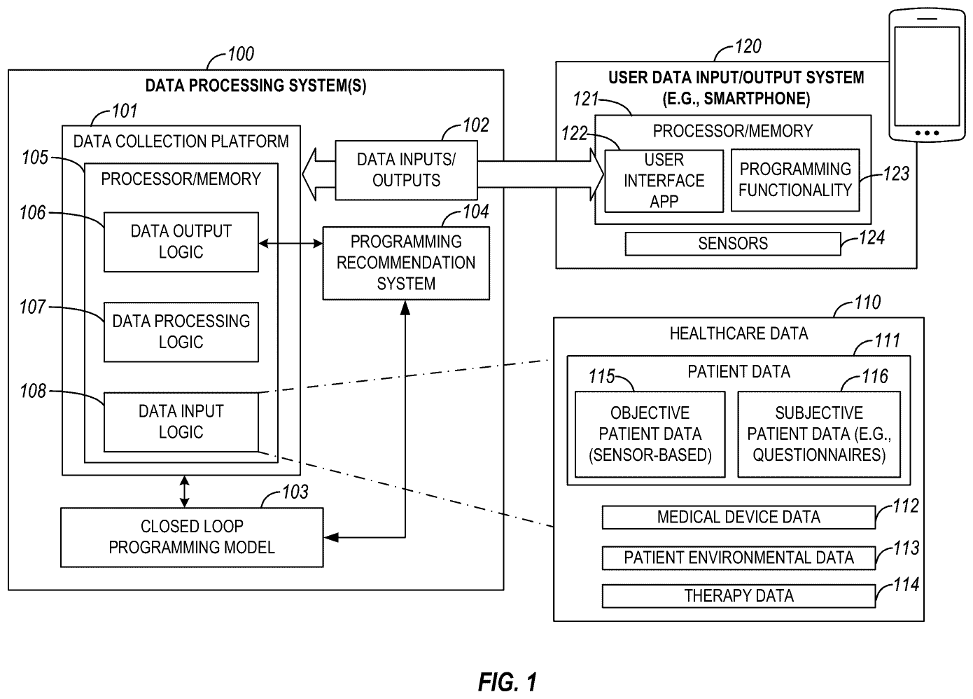

FIG. 1 illustrates, by way of example, an embodiment of a data processing system 100 configured to analyze data and generate recommendations in connection with the use of neurostimulation programs and closed-loop neurostimulation programming. The illustrated data processing system 100 is configured to include at least one data collection platform 101, which operates to collect and provide data inputs/outputs 102. The data collection platform 101 is configured to process (e.g., filter, extract, transform) the input data to produce data to be used for one or more iterations of closed-loop programming with a closed-loop programming model 103. The closed-loop programming model 103 may generate various programming changes or adaptations that can be suggested to a user via a programming recommendation system 104. The data collection platform 101 can also generate output data in the form of guidance, suggestions, and other information which is relevant to programming activities, and the collection of feedback or other inputs.

The data processing system 100 may be implemented at one or more server(s) or other systems remotely located from the patient. The data processing system 100 may use various network protocols to communicate and transfer data through one or more networks such as the Internet. The data processing system 100 and data collection platform 101 may include at least one processor configured to execute instructions stored in memory (e.g., depicted as processor(s)/memory 105) to generate or evaluate data outputs 106, to obtain or evaluate data inputs 108, and to perform data processing 107 on both inputs and outputs and accompanying data. For instance, the data inputs 108 may be processed to generate programming data with a closed-loop programming model 103, to identify a recommended programming change or setting based on actual patient usage (e.g., sensor observations) and patient feedback (e.g., feedback that indicates whether the settings have been effective or have not been effective).

The data inputs 108 may include information obtained from the patient directly, and may include different types of healthcare data 110 associated with the patient or the treatment. Examples of healthcare data 110 may include patient data 111, medical device data 112, patient environmental data 113, therapy data 114, or various combinations thereof. The patient data 111 may include objective data 115 such as data collected from physiological sensor(s) and subjective data 116 such as data collected from user-answered question(s) (e.g., “How do you rate your pain?”). Objective and subjective data may be provided by the patient, a caregiver, a clinician, or another third party (device manufacturer or service provider). Objective data, as used herein, is data that can obtained from a measurement or direct observation. Objective data may be measured by a sensor and may be provided via user input when the user has access to objectively determined information. Categories of objective data may include physiological parameter data, therapy data, device data, and environmental data. By way of example and not limitation, physical parameter data may include data such as: heart rate, blood pressure, respiration rate, activity, posture, electromyograms (EMGs), neural responses such as evoked compound action potentials (ecaps), glucose measurements, oxygen levels body temperature, oxygen saturation and gait. By way of example and not limitation, therapy data may include: neuromodulation programs, therapy on/off schedule, dosing, neuromodulation parameters such as waveform, frequency, amplitude, pulse width, period, therapy usage and therapy type. By way of example and not limitation, device data may include: battery information (voltage, charge state, charging history if rechargeable), impedance data, faults, device model, lead models, MRI status, Bluetooth connection logs, connection history with Clinician's Programmer (CP). By way of example and not limitation, environmental data may include: temperature, air quality, pressure, location, altitude, sunny, cloudy, precipitation, etc. Subjective data can include information received from the patient or another human user (e.g., caregiver, clinician, etc.). For example, the patient's quantification of pain can provide subjective data. Subjective data may generally involve user-inputted data. Examples of subjective data include questions with free text answers, multiple choice questions, question tree(s), and different question subject(s). Other data may be stored and/or transferred, including detected event data to track events (e.g., that trigger a response, change data resolution), contextual data, time data, and the like. The event(s), context(s) and time may be detected by the system or may be provided via user input.

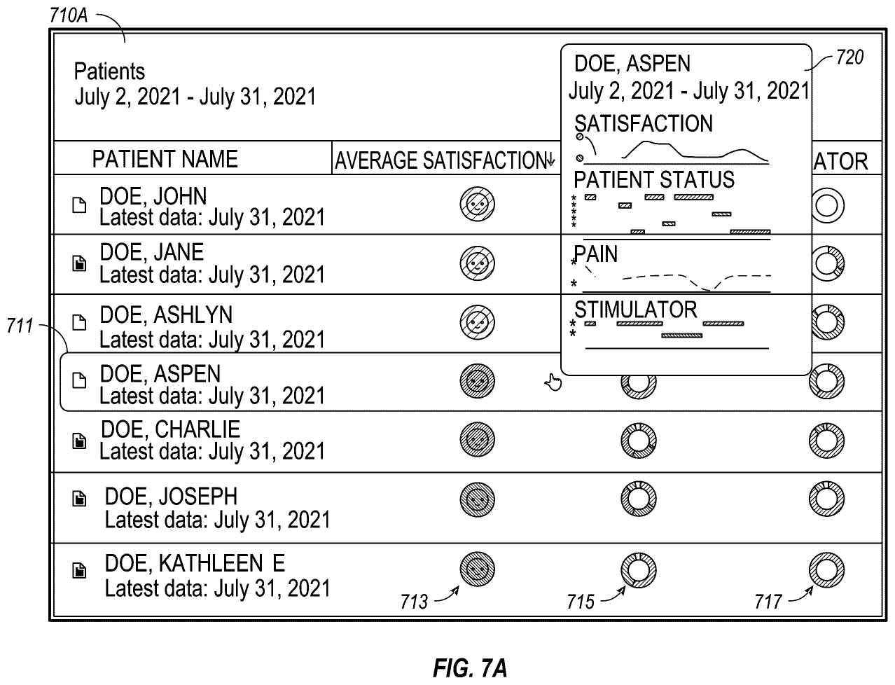

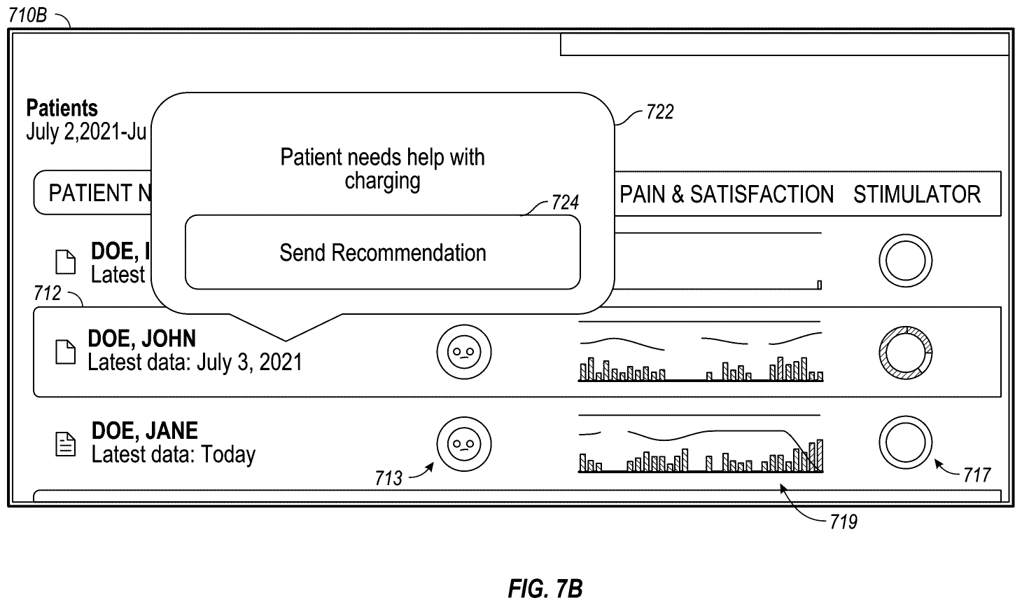

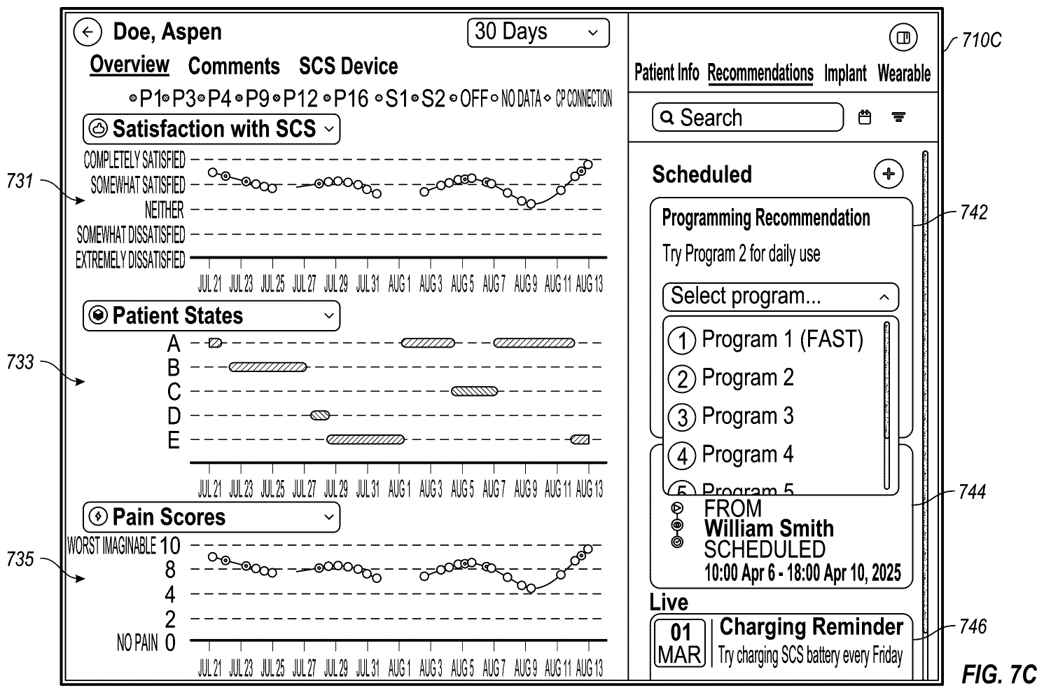

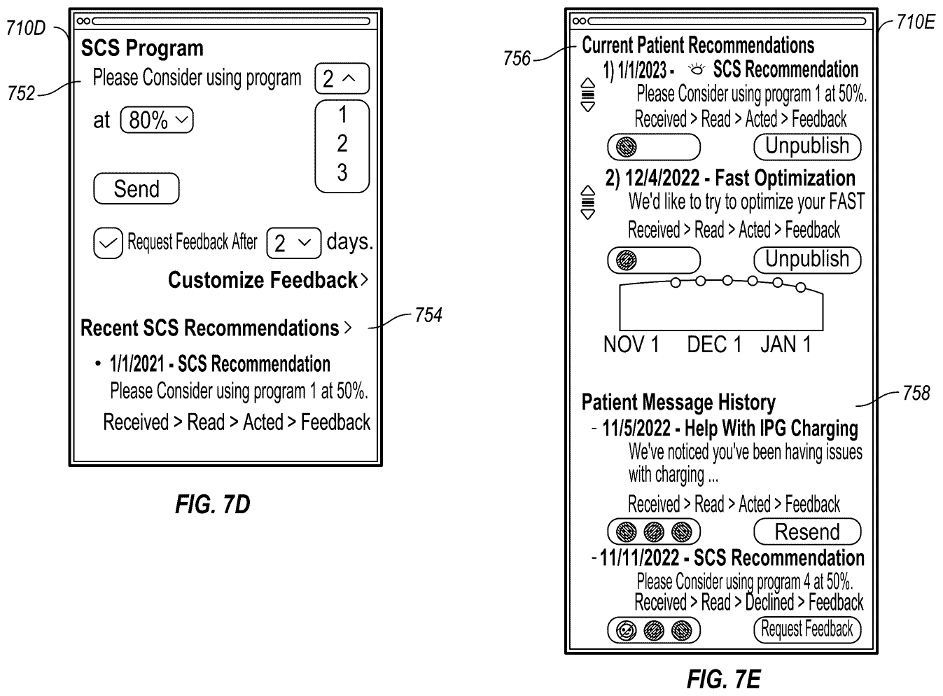

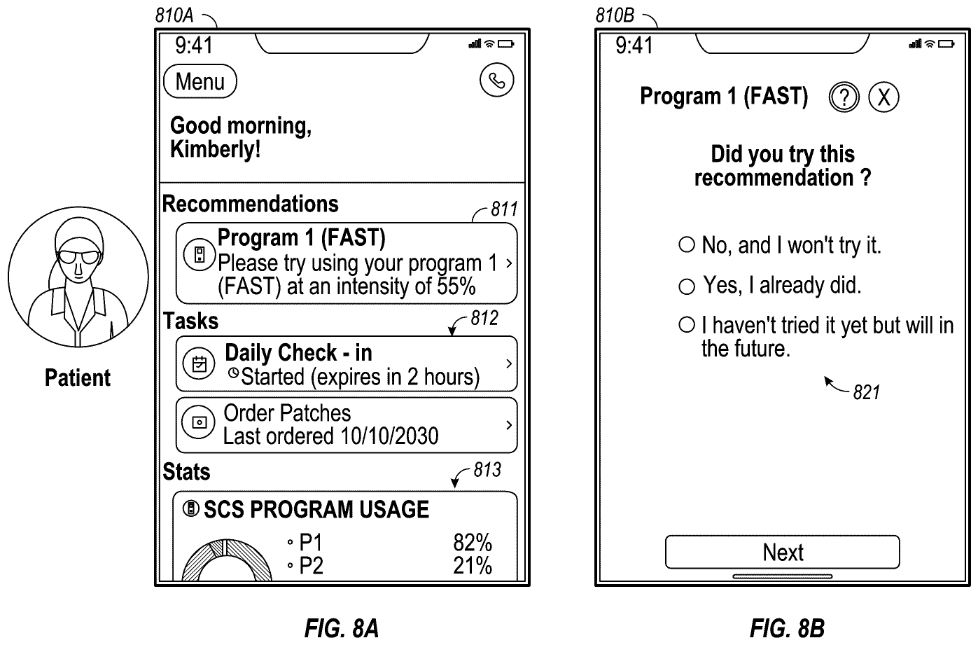

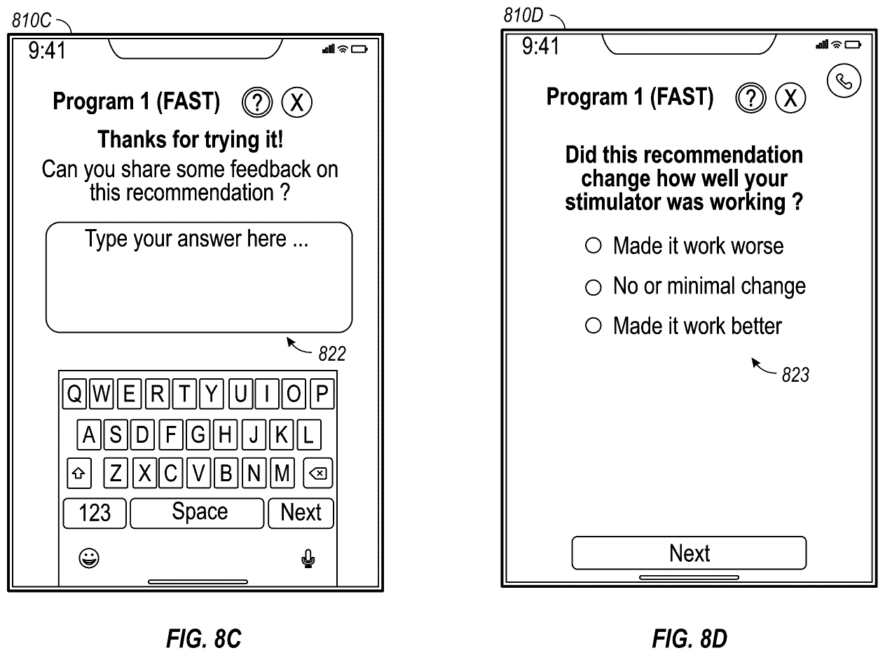

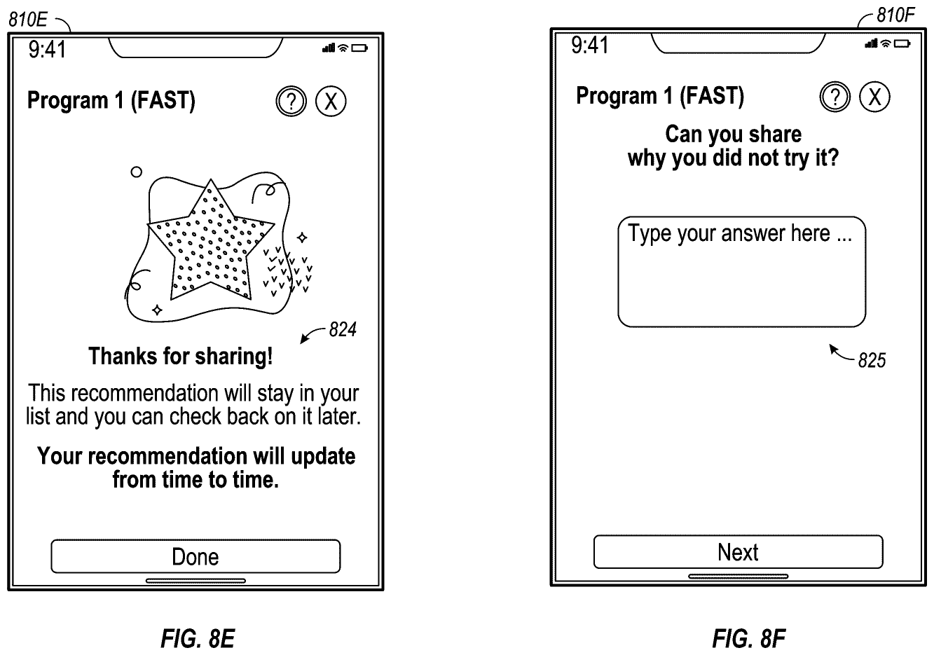

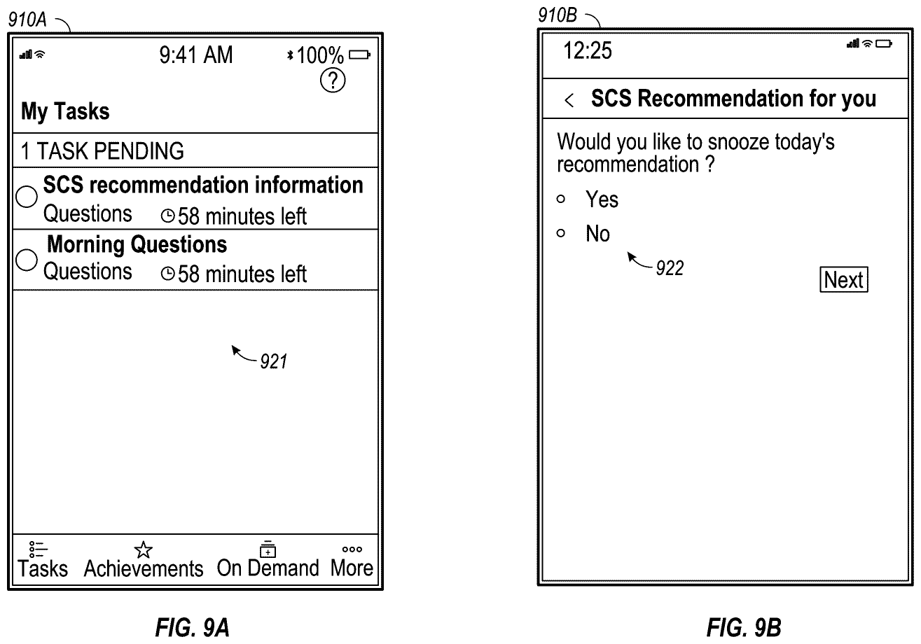

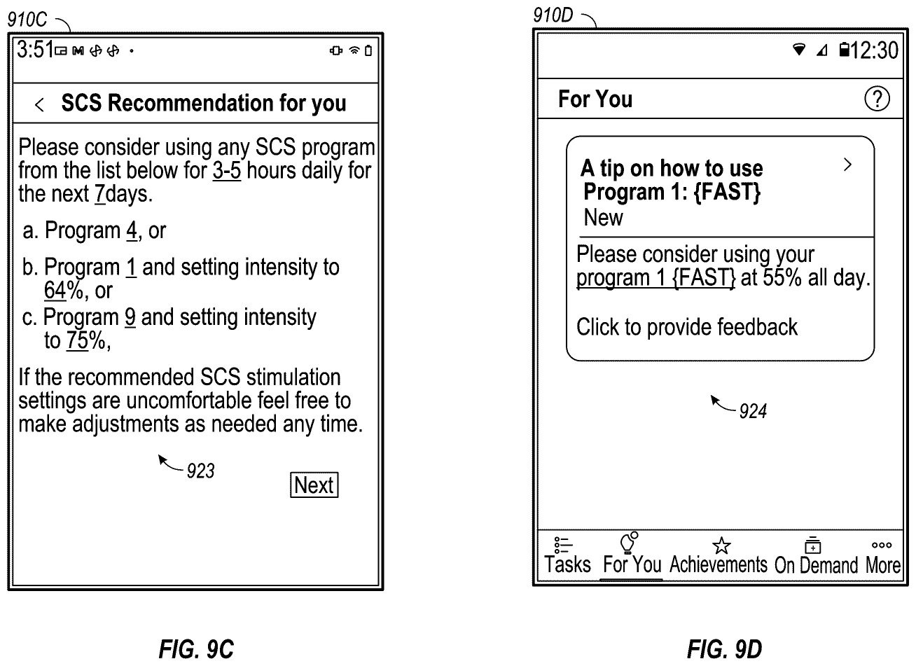

The user data input/output system 120 may be implemented at one or more devices located at or operated by the patient, such as via a smartphone, personal computer, tablet, smart home device, a remote programmer, a programming device, or another compute device or platform capable of collecting input and providing output. The user data input/output system 120 may include at least one processor configured to execute instructions stored in memory (e.g., depicted as processor(s)/memory 121) to provide data input(s) and outputs relevant to implementing closed-loop programming recommendations, and providing feedback and control of related neurostimulation device programming. One such example is a user interface application 122 implemented as a graphical user interface (GUI), that provides commands to programming functionality 123. Examples of patient GUIs for data inputs and outputs related to recommendations and programming include the patient smartphone apps illustrated in FIGS. 8A-8F and FIGS. 9A-9D, discussed below. Examples of medical user GUIs for data inputs and outputs related to recommendations and programming include the medical user dashboard screens illustrated in FIGS. 7A-7E, discussed below.

The user data input/output system 120 may include or interface with sensors 124 such as physiological sensors that can detect patient state information (e.g., activity, sleep), event(s), indicators of device usage, patient compliance with data collection and/or therapy, or various combinations thereof. The user data input/output system 120 may directly or indirectly capture measurements from the sensors 124 or from associated external systems. Examples of external system(s) include remote controls, programmers, phones, tablets, smart watches, personal computers, and the like. The external system may be configured to provide the medical device data 112, the patient environmental data 113, or the therapy data 114 from an associated medical device.

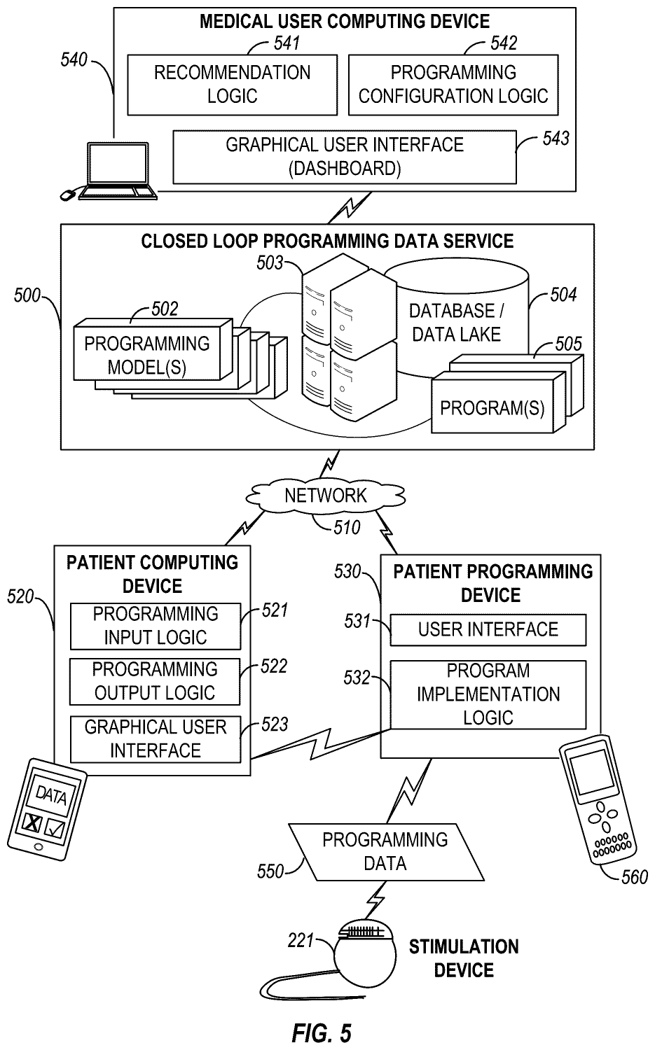

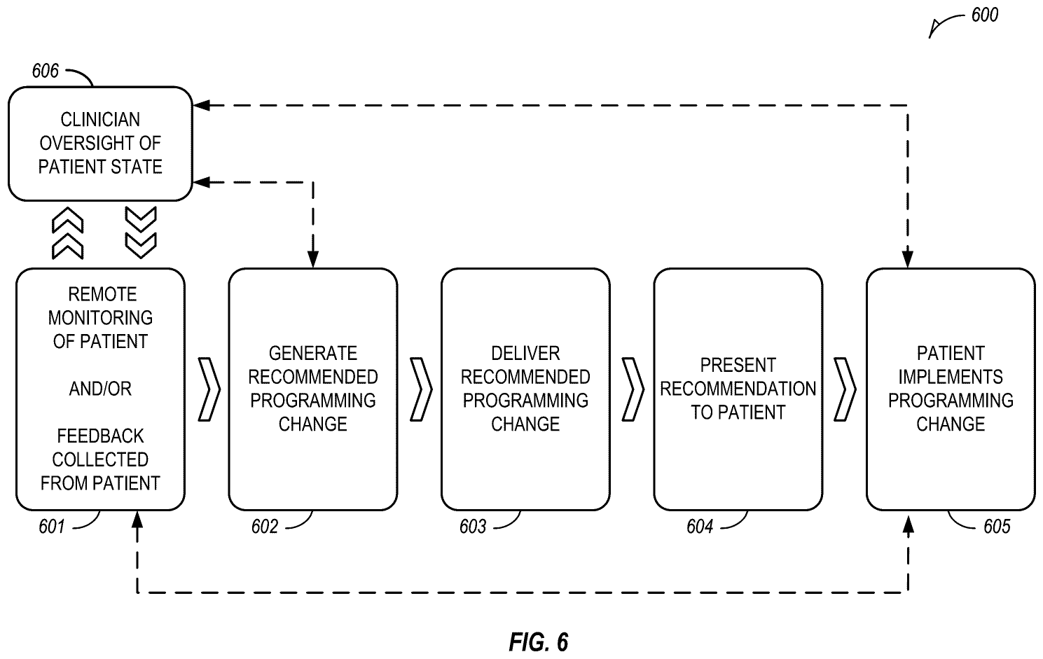

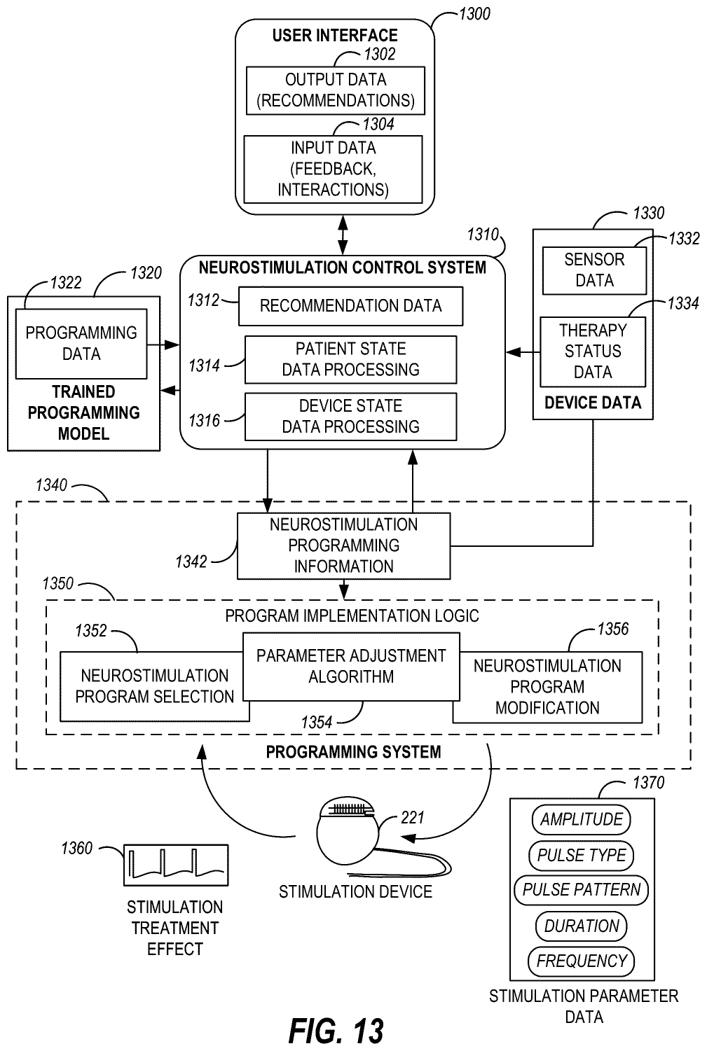

The closed-loop programming model 103 may operate a programming model (e.g., an artificial intelligence model, such as a neural network) that has been trained to infer or generate new programs and program parameters. One or multiple instances of a model may be trained to generate programs and program parameters, with the use of one or multiple patient-specific or population-based models. The closed-loop programming model 103 may consider multiple aspects (and, dynamic aspects) of patient-specific or population data such as the healthcare data 110, sensor data, and rules and information from a variety of data sources. More details on an example closed-loop programming workflow is depicted in FIG. 5, FIG. 6, and FIG. 13 discussed below.

In some examples, the data collection platform 101 and the data processing system 100 may directly interface with one or more medical device(s), external system(s) or other healthcare related data source(s) to collect the healthcare data 110. One or more of the medical device(s), external system(s) or other healthcare-related data source(s) may include technology used by the data processing system 100 to collect data, and thus may form part of the data collection platform 101. Examples of medical devices include implantable and wearable devices. The medical device may be configured to only collect data, to only deliver therapy, or to both collect data and deliver therapy. The medical device may be configured to collect and provide medical device data such as device model, configuration, settings, and the like. Thus, the medical device may provide the patient data 111, the medical device data 112, the patient environmental data 113, and therapy data 114, particularly in specialized neurostimulation programming environments.

Other healthcare-related data source(s) may include patient data received via a provider's server that stores patient health records (e.g., test results, doctor notes, prescriptions, and the like). Other healthcare-related data sources may include apps on a patient's smartphone or other computing device, or the data on a server accessed by those apps. By way of example and not limitation, this type of data may include heart rate, blood pressure, weight, and the like collected by the patient in their home. In another example, an app on a phone or patient's device may include or may be configured to access environmental data such as weather data and air quality information or location elevation data. Weather data may include, but is not limited to, barometric pressure, temperature, sunny or cloud cover, wind speed, and the like.

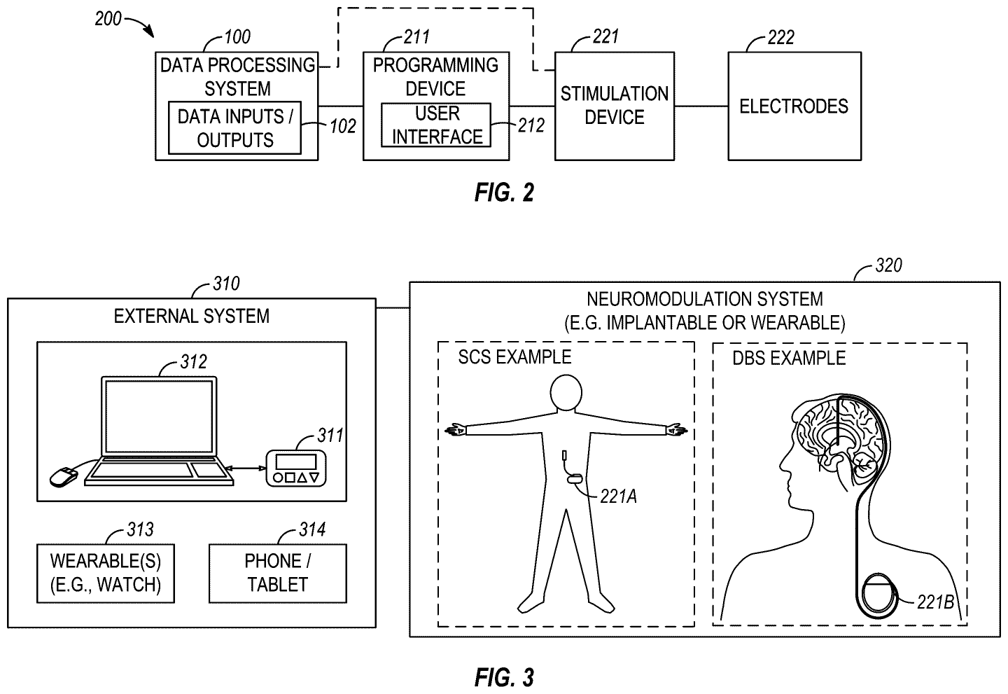

FIG. 2 illustrates a neuromodulation system as an example of a medical device system. The medical device may be configured, by way of example and not limitation, to deliver an electrical therapy using one or more electrodes 222 provided by a stimulation device 221. In the illustrated embodiment, the medical device may be a neurostimulation device, and the system may be a neuromodulation system 200.

The illustrated neuromodulation system 200 includes electrodes 222, the stimulation device 221 and a programming system such as a programming device 211. The programming system may include multiple devices. The electrodes 222 are configured to be placed on or near one or more neural targets in a patient. The stimulation device 221 is configured to be electrically connected to the electrodes 222 and deliver neuromodulation energy, such as in the form of electrical pulses, to the one or more neural targets though the electrodes 222. The system may also include sensing circuitry to sense a physiological signal, which may but does not necessarily form a part of stimulation device 221. The delivery of the neuromodulation is controlled using a plurality of modulation parameters that may specify the electrical waveform (e.g., pulses or pulse patterns or other waveform shapes) and a selection of electrodes through which the electrical waveform is delivered. In various embodiments, at least some parameters of the plurality of modulation parameters are programmable by a user, such as a physician or other caregiver, using one or more program. The programming device 211 thus provides the user with accessibility to the user-programmable parameters. The programming device 211 may also provide the use with data indicative of the sensed physiological signal or feature(s) of the sensed physiological signal.

In various embodiments, the programming device 211 is configured to be communicatively coupled to the stimulation device 221 via a wired or wireless link. In various embodiments, the programming device 211 includes a user interface 212 such as a graphical user interface (GUI) that allows the user to set and/or adjust values of the user-programmable modulation parameters, including parameters provided via recommendations as discussed herein. The user interface 212 may also allow the user to view the data indicative of the sensed physiological signal or feature(s) of the sensed physiological signal and may allow the user to compare the sensed data to expected or recommended data values. The data may be provided to the data processing system 100, which processes data inputs and outputs 102 to assist the user with the operation, training, configuration, maintenance, or improvement of the stimulation device 221.

The user interface 212 of the programming device 211 may be used to allow the user to answer questions to provide healthcare-related data, although other devices (such as a patient smartphone) may be used as discussed below. The user interface 212 of the programming device 211 may also include the ability to receive or display recommendations, such as recommendations downloaded from a network from other local devices (such as a patient smartphone). Therapy parameters, programming selection, electrode selection, and other operational parameters may also provide healthcare-related data for use by the data processing system 100. Additional sensor(s) may also provide healthcare-related data for use by the data processing system.

FIG. 3 illustrates, by way of example and not limitation, the neurostimulation system of FIG. 2 implemented in a spinal cord stimulation (SCS) system or a deep brain stimulation (DBS) system. The illustrated neuromodulation system 320 connects with an external system 310 that may include at least one programming device. The illustrated external system 310 may include a programmer 312 (e.g., clinician programmer) configured for use by a clinician to communicate with and program the neurostimulator, and a remote control device 311 configured for use by the patient to communicate with and program the neurostimulator. For example, the remote control device 311 may allow the patient to turn a therapy on and off and/or may allow the patient to adjust patient-programmable parameter(s) of the plurality of modulation parameters (e.g., by switching programs).

FIG. 3 further illustrates the neuromodulation system 320 as an ambulatory medical device, such as implemented by stimulation device 221A or stimulation device 221B. Examples of ambulatory devices include wearable or implantable neuromodulators. The external system 310 may include a network of computers, including computer(s) remotely located from the ambulatory medical device that are capable of communicating via one or more communication networks with the programmer 312 and/or the remote control device 311. The remotely located computer(s) and the ambulatory medical device may be configured to communicate with each other via another external device such as the programmer 312 or the remote control device 311.

The external system 310 may also include one or more wearables 313 and a portable device 314 such as a smartphone or tablet. In some examples, the wearables 313 and the portable device 314 may allow a user to obtain and provide input data, such as sensor data values (e.g., from a physiologic sensor of a wearable) or feedback/status information (e.g., on a phone/tablet screen) in connection with a data collection process. In some examples, the remote control device 311 and/or the programmer 312 also may display recommendations or program settings derived from recommendations as part of a partially closed-loop programming process. The remote control device 311 and/or the programmer 312 may be used to communicate other aspects of input and output, including inputs from the usage data of various neurostimulation programs, events associated with such programs, and the like.

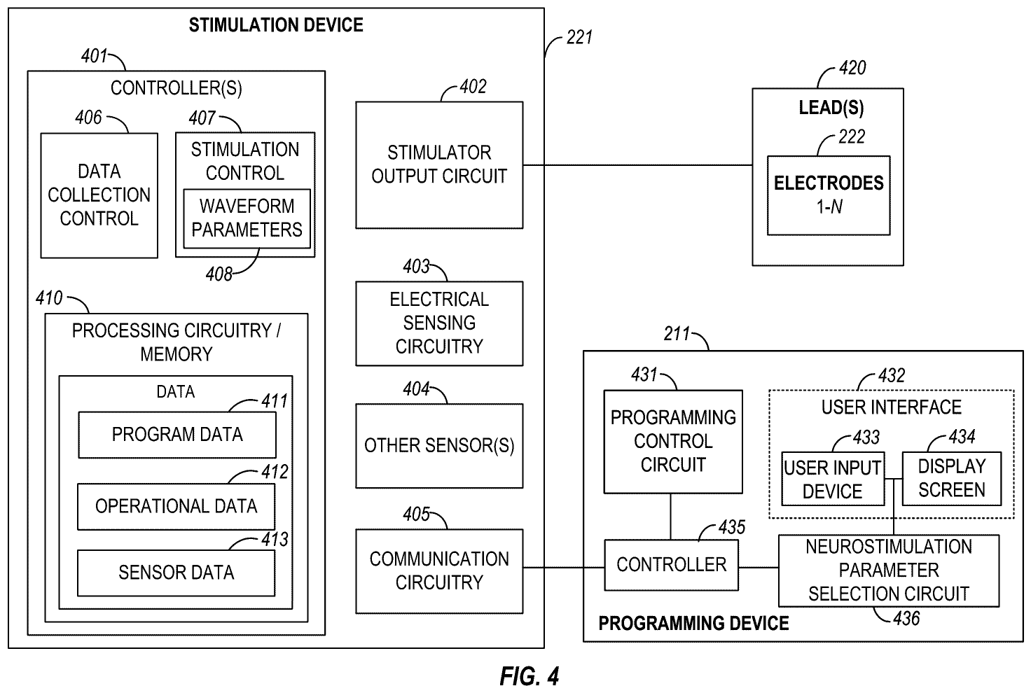

FIG. 4 illustrates, by way of example, an embodiment of a neuromodulation device, such as may be implemented as the stimulation device 221 illustrated in FIGS. 2 and 3. The stimulation device 221 may be configured to be connected to electrode(s) 222, illustrated as N electrodes, via one or more leads 420. Any one or more of the electrodes 222 may be configured for use to deliver modulation energy, sense electrical activity, or both deliver modulation energy and sense electrical activity. The stimulation device 221 may include a stimulator output circuit 402 configured to deliver modulation energy to electrode(s). The stimulator output circuit 402 may be configured with multiple (e.g., two or more) channels for delivering modulation energy, where each channel may be independently controlled with respect to other channel(s). For example, the stimulator output circuit 402 may have independent sources such as independent current sources or independent voltage sources.

In various examples, the electrodes 222 may include a stimulation electrode or a sensing electrode. The stimulation electrode is configured for use in delivering modulation energy, and the sensing electrode is configured for use in sensing electrical activity. As illustrated, the stimulation electrode may also be used in sensing electrical activity, and the sensing electrode may also be used in delivering modulation energy. Thus, the term “stimulation electrode” does not necessary exclude the electrode from also being used to sense electrical activity; and the term “sensing electrode” does not necessarily exclude the electrode from also being used to deliver modulation energy.

The stimulation device 221 may include electrical sensing circuitry 403 configured to receive sensed electrical energy from the electrode(s), such as may be used to sense electrical activity in neural tissue or muscle tissue. The sensing circuitry may be configured to process signals in multiple (e.g., two or more) channels. By way of example and not limitation, the electrical sensing circuitry 403 may be configured to amplify and filter the signal(s) in the channel(s).

The controller 401 may be configured to detect one or more features in the sensed signals. Examples of features that may be detected include peaks (e.g., minimum and/or maximum peaks including local peaks/inflections), range between minimum/maximum peaks, local minima and/or local maxima, area under the curve (AUC), curve length between points in the curve, oscillation frequency, rate of decay after a peak, a difference between features, and a feature change with respect to a baseline. Detected feature(s) may be fed into a control algorithm, which may use relationship(s) between the feature(s) and waveform parameter(s) to determine feedback for closed-loop control of the therapy. Some embodiments of the stimulation device 221 may include or be configured to receive data from other sensor(s) 404. The other sensor(s) 404 may include physiological sensor(s), environmental sensor(s), or proximity sensor(s).

The stimulation device 221 may include a controller 401 operably connected to the stimulator output circuit 402 and the electrical sensing circuitry 403. The controller 401 may include a stimulation control 407 (e.g., logic) configured for controlling the stimulator output circuit 402. For example, the stimulation control 407 may include start/stop information for the stimulation and/or may include relative timing information between stimulation channels. The stimulation control 407 may include waveform parameters 408 (e.g., associated with a program or a defined set of parameters) that control the waveform characteristics of the waveform produced by the stimulator output circuit 402. The waveform parameters 408 may include, by way of example and not limitation, amplitude, frequency, and pulse width parameters. The waveform parameters 408 may include, by way of example and not limitation, regular patterns such as patterns regularly repeat with same pulse-to-pulse interval and./or irregular patterns of pulses such as patterns with variable pulse-to-pulse intervals. The waveform parameters may, but do not necessary, define more than one waveform shape (e.g., including a shape other than square pulses with different widths or amplitudes). The stimulation control 407 may be configured to change waveform parameter(s) (e.g., one or more waveform parameters) in response to user input and/or automatically in response to feedback.

The controller 401 may include a data collection control 406 configured for use by the stimulation device 221, and the data collection platform 101 of a data processing system 100 (see FIGS. 1-2), to collect healthcare related data. The controller 401 may include a processor and/or memory 410 (e.g., with instructions) for use to control the data collection using the data collection control 406 and control the stimulation via the stimulation control 407. The memory may also provide storage for storing different types of collected healthcare-related data, such as program data 411, operational data 412, sensor data 413, and the like. The program data 411 and operational data 412 may be provided or implemented as a result of the closed-loop programming recommendations discussed herein. Examples of sensor data 413 collected by the stimulation device 221 or other devices discussed herein may include, by way of example and not limitation, heart rate, heart rate variability, oxygen saturation, activity, posture, steps, gait, temperature, evoked compound action potentials (ECAPS), electromyograms (EMGs), electroencephalograms (EEGs), weight, blood pressure, and the like. Examples of program data 411 may include, by way of example and not limitation, stimulation settings such as amplitude, pulse width, pulse frequency period, duration of burst of pulses, active electrodes, electrode fractionalization controlling the distribution of energy (e.g., current) to active electrodes, waveforms, pulse patterns including various complex patterns, and the like. Examples of operational data 412 of the stimulation device 221 may include, by way of example and not limitation, electrode-tissue impedance, fault conditions, battery information such as battery health, battery life, voltage, charge state, charging history if rechargeable, MRI status, Bluetooth connection logs, connection with a clinician programmer, hours of operation/duration of implant, and the like. Other device information may include device model and lead model.

The neuromodulation device may include communication circuitry 405 configured for use to communicate with other device(s) such as a programming device 211, remote control, phone, tablet and the like. The healthcare-related data may be transferred out from the neuromodulation device for transfer to a data processing system, as discussed above. As shown, a programming device 211 includes a programming control circuit 431, a user interface 432 (e.g., including a user input device 433 such as a buttons and a display screen 434), a controller 435, and other components (e.g., an external communication device, not shown) to effect programming of a connected neurostimulation device. The operation of the neurostimulation parameter selection circuit 436 enables selection, modification, and implementation of a particular set of parameters or settings for neurostimulation programming (e.g., via selection of a program, implementation of a closed-loop or open-loop programming process, specification by a patient or clinician, or the like).

FIG. 5 illustrates, by way of example, an embodiment of data interactions among a data processing system implemented as a closed-loop programming data service 500, for operation of a stimulation device 221 with use of closed-loop programming, including partially-automated or human-directed closed-loop programming approaches, also referred to as a “partially closed-loop” system. As used herein, a “partially closed-loop” system refers to use of a closed-loop system that automatically generates or suggests programming values, but also relies on some form of clinician, patient, or third party intervention which customizes the settings, timing, or use of the programming values.

At a high level, a closed-loop programming data service 500 uses a trained model (one of programming models 502) to generate recommended programming settings and parameters (e.g., provided in one or more programs 505) that are customized to the human patient. The closed-loop programming data service 500 includes compute hardware 503 to control model inference and training and other data processing operations, and to generate or control diagnostic actions, alerts, programming recommendations, or programming actions. The programming settings and parameters may be implemented automatically or manually on the stimulation device 221 based on implementing recommendations or actions via a patient computing device 520 or patient programming device 530.

The closed-loop programming data service 500 communicates with one or both of the patient computing device 520 or patient programming device 530, to obtain data for analysis by the programming models 502. The data may be stored in a database 504 or another large-scale data store (e.g. data lake) for the patient or a population of patients. The closed-loop programming data service 500 may also include data analysis or processing engines (not shown) that parse and determine a state of a particular patient from various inputs and correlated program usage (e.g., to determine what programs and programming settings are beneficial or not beneficial to the patient). In some examples, the state of treatment may be based on correlating the historical use of a neurostimulation program or set of parameters with the current state of a patient (e.g., identifying that a pain condition became worse or better after beginning use of a particular program). Other forms of closed-loop programming may involve the analysis of user (medical user or patient) actions, physiological results, and behavioral results, to automatically select or guide the programming based on detected conditions.

The closed-loop programming data service 500 may also analyze a variety of forms of patient input and patient data related to usage of a neurostimulation program or neurostimulation programming parameters. For instance, the closed-loop programming data service 500 may receive information from program usage, survey inputs, or even text input originating from a human patient, via the patient computing device 520 and patient programming device 530. The closed-loop programming data service 500 may also utilize sensor data from one or more patient sensors (e.g., wearables, sleep trackers, motion tracker, implantable devices, etc.) among one or more internal or external devices. The sensor data may be used to determine a customized and current state of the patient condition or neurostimulation treatment results.

The closed-loop programming data service 500 may be controlled (at least in part) by a medical user computing device 540, which enables a doctor, medical device company representative, or other medical professional to oversee the types of programming recommendations and effects at the patient's stimulation device. In an example, the medical user computing device 540 hosts a graphical user interface 543 that provides a dashboard for control of closed-loop programming recommendations, and a display of patient statuses and information. The medical user computing device 540 also hosts recommendation logic 541 that is used to customize recommendations of suggested programming changes, and programming configuration logic 542 that is used to suggest and cause particular programming data values and operational settings to be delivered to a patient or group of patients.

In addition to recommending programs and program settings, the closed-loop programming data service 500 may provide other therapy content and recommendations to the patient computing device 520 and patient programming device 530. In an example, the patient computing device 520 is a computing device (e.g., personal computer, tablet, smartphone) or other form of user-interactive device which receives and provides interaction with a patient using a graphical user interface 523, implementing programming input logic 521 and programming output logic 522. For instance, the programming input logic 521 may receive input from a patient via questionnaires, surveys, messaging, or other inputs. The inputs may provide text related to pain or satisfaction, which can be used to identify a psychological or physiological state of the patient, neurostimulation treatment results, or related conditions.

A patient programming device 530 is depicted as including a user interface 531 and program implementation logic 532. The program implementation logic 532 specifically may provide the patient with the ability to implement or switch to particular programs and recommendations such as those generated by closed-loop programming data service 500. The patient programming device 530 communicates programming data 550 in the form of settings, programs, and data values to the stimulation device. In some examples, the patient programming device 530 directly communicates with the closed-loop programming data service 500 via a network. In other examples (such as the QR code data transfer discussed below), the closed-loop programming data service 500 provides programming settings to the patient computing device 520 which then communicates the recommended programming settings to the patient programming device 530. In still other examples, instructions on how to implement the recommended programming settings are displayed to the user (e.g., via the graphical user interface 523) and the user enters the settings manually into the patient programming device 530 via the user interface 531.

In various examples, the stimulation device 221 also includes sensors that contribute to the sensor data to be evaluated by the closed-loop programming data service 500. In an example, such sensors are physiological or biopsychosocial sensors that collect data relevant to physical, biopsychosocial (e.g., stress and/or mood biomarkers), or physiological factors relevant to a state of the patient. Examples of such sensors might include a sleep sensor to sense the patient's sleep state (e.g., for detecting lack of sleep), a respiration sensor to measure patient breathing rate or capacity, a movement sensor to identify an amount or type of movement, a heart rate sensor to sense the patient's heart rate, a blood pressure sensor to sense the patient's blood pressure, an electrodermal activity (EDA) sensor to sense the patient's EDA (e.g., galvanic skin response), a facial recognition sensor to sense the patient's facial expression, a voice sensor (e.g., microphone) to sense the patient's voice, and/or an electrochemical sensor to sense stress biomarkers from the patient's body fluids (e.g., enzymes and/or ions, such as lactate or cortisol from saliva or sweat). Other types or form factors of sensor devices may also be utilized.

FIG. 6 depicts a workflow 600 for integrating medical user or patient actions into closed-loop programming of a neuromodulation system, such as the stimulation device 221 discussed above. It will be understood that additional user or automated actions, commands, and data operations may be involved as part of closed-loop programming, and are omitted for purposes of simplicity.

At 601, the workflow begins with data collection, provided from the remote monitoring of a patient and/or feedback collected from a patient. Such data collection may involve the monitoring of patient state and activities such as pain, sleep, medication, mental state or mood, and physical state or activity. Additionally, or alternatively, the data collection may include feedback information (e.g., survey answers, freeform text, etc.) that is provided from a patient, which indicates whether a particular treatment is or is not working, or whether a patient's condition is or is not improving.

At 602, the workflow continues with the generation of a recommended programming change. The recommended programming change may be generated by programming models, rules, or artificial intelligence algorithms, and may be customized based on patient-specific or population rules. The recommended programming change may relate to simple changes (e.g., how long that a neurostimulation program is used, or whether to use a different program) or detailed changes (e.g., new waveforms to use in a new program, waveform settings or changes, etc.).

At 603, the workflow continues with the delivery of the recommended programming change to a patient device (e.g., patient computing device 520 or patient programming device 530, or both). This may be provided in the form of program settings or program data, suggestions, instructions, informational steps, or commands that are directly or indirectly delivered to a patient device.

At 604, the recommended programming change is presented to the patient via the patient device. This recommended programming change may be accompanied by user interface options such as to immediately implement the programming change, schedule or defer the programming change, to reject or modify the programming change, or to display the status of the programming change. Examples of these recommended programming changes is depicted among FIGS. 8A to 10C.

At 605, if the recommended programming change is implemented, then one or more neurostimulation programs (e.g., new programs, modified programs) are deployed to the stimulation device. The programs may be automatically scheduled or modified, or the programs may be made available for selection by the patient. The later use of the recommended programming change (or, patient or medical user/clinician actions taken based on the recommendation) may be used to collect additional feedback and the closed-loop improvement. Additionally, the later use of the recommended programming change may be communicated to a medical user or third party.

At 606, oversight is provided by a clinician or third party (e.g., caregiver, device manufacturer representative, etc.), based on the implementation or non-implementation of the recommendation, and any effects that are observed after the use of the programming change. As a result, closed-loop improvements may be provided to subsequent programming changes. In other examples, the oversight that is provided by a clinician or third party may occur before specific recommendations are generated and delivered.

The next examples include user interfaces and interactions to control the generation and presentation of closed-loop programming recommendations, including in clinical and patient settings. It will be understood that the data processing performed by the present approaches may occur on a variety of user-interactive interfaces, including text, graphical, or voice inputs and outputs, and are not limited to the screens discussed below.

FIGS. 7A, 7B, 7C, 7D, and 7E depict user interfaces 710A, 710B, 710C, 710D, 710E (e.g., a website graphical user interface) that provide output on a medical user device (e.g., a personal computing device, workstation, tablet, or smartphone, operated by a medical professional or authorized user). For instance, these user interfaces may be operated by a clinician or care team using a website or installed app to suggest and control delivery of a recommendation for a patient. The user interfaces 710A, 710B, 710C, 710D, 710E may provide an implementation of the graphical user interface 543 and the dashboard functionality discussed above.

Within the user interfaces 710A, 710B, 710C, 710D, 710E, a general workflow may be provided as follows. First, the clinician/care team reviews patient data. Next, the clinician/care team chooses a recommendation from a list of recommendations (e.g., recommendations automatically selected by a closed-loop programming model or algorithm). The clinician/care team customizes or modifies the recommendation or programming settings associated with the recommendation, such as by configuring recommendation parameters (e.g., program recommendations, waveform settings, etc.). The clinician/care team sends the recommendation and programming settings in one or more messages to one or more patient(s). The recommendation and programming settings are delivered to a patient device (e.g., a patient programming app that displays basic controls for using a program, amplitude settings, and on-off settings), allowing the patient to choose whether to follow the recommendation or ignore the recommendation. More details on the patient interaction are shown in FIGS. 8A-9D.

FIG. 7A depicts the data input user interface 710A as including a listing of patients in a dashboard screen. This listing of patients includes information relevant to treatment of each patient, such as a satisfaction rating 713, a pain rating 715, and a stimulator operation rating 717 for each patient (e.g., with rating values represented with icons, colors, shading, etc.). The data input user interface 710A also presents a detailed view 720 of a selected patient 711, allowing a clinician/care team user to view graphs or other graphical representations of neurostimulation data or patient state data collected over a period of time.

FIG. 7B depicts the data input user interface 710B as including another view of data from patients including another selected patient 712, and presenting the satisfaction rating 713, the stimulator operation rating 717, and an expanded graphical chart 719 of pain and satisfaction values over time. As depicted, an overlay pop-up message is displayed for the selected patient 712, indicating that the patient needs help with charging. A selectable user interface control (e.g., button) may be activated to communicate a recommendation that provides helpful assistance and instruction to the user regarding charging.

FIG. 7C depicts the data input user interface 710C as including a detailed view of data from a selected patient, including a graphical chart 731 with data values for satisfaction (charted over a time period), a graphical chart 733 with data values for patient states (charted over the same time period), and a graphical chart 735 with data values for pain scores (charted over the same time period). The data input user interface 710C also includes a suggested programming recommendation 742 and programming recommendation schedule 744, which allows timing of the future recommendations to be customized or changed by the clinician/care team user. The data input user interface 710C also displays a list of live recommendations 746 that are being or have been presented to the patient.

FIG. 7D depicts the data input user interface 710D as including a summarized screen to customize a delivery of a recommendation. This data input user interface 710D includes program settings 752 that allow the selection of a program, an amplitude value for the program, and a requested feedback for the use of the program. The data input user interface 710D also includes a listing of recent recommendations 754 to inform the clinician/care team user about previous attempts for programming.

FIG. 7E similarly depicts the data input user interface 710E as including a listing of current patient recommendations 756, and a listing of patient messaging history 758 used to deliver patient recommendations. As will be understood, the data input user interfaces 710D and 710E support additional aspects of creating, customizing, configuring, sending and viewing recommendations and recommendation settings, and requests for feedback messages and content from patients.

Other features of a dashboard or clinician/care team interface, not shown, can include: enabling a clinician/care team user to select a recommendation from a closed-list of feedback messages with optional parameter configuration; and providing a graphical representation of a life cycle of a recommendation/message (e.g., when created and sent, received by a patient, completed or not completed, and what feedback was provided). In further examples, other user interactions provided by a dashboard or clinician/care team interface may include: presenting an ability to cancel/remove a recommendation, override a recommendation, or prioritize recommendations; displaying outcomes that display past messages and patient feedback; viewing a history of previously sent messages, including if/how the patient received the message, how/when the patient interacted with message, any feedback that the patient provided, and backend/event data associated with the message.

Other types of closed-loop programming logic and data analysis may assist the use of a clinician/care team interface. Messages can be suggested based on the patient's data (e.g., a patient state is worsening, so a list of messages with ‘improve outcomes’ messages are presented at the top of the list for use). Messages also may be recommended to be delivered in a partially-automated manner. For instance, a message author can specify when/what feedback is required from the patient user. Messages may also be automatically tracked, as a system sends scheduled/configured feedback campaign to user, and associated with a unique ID for the message or campaign.

Feedback for delivered recommendations may be provided by any combination of voice, freeform text, or answers to formatted questions. In some examples, input data may be provided from non-graphical or non-text inputs, such as voice interactions controlled by a chatbot or by virtual assistants or agents (e.g., Amazon® Alexa, Google® Assistant, Apple® Siri, etc. Further, any of the user interfaces discussed herein may include other user input functionality, such as menu selection functionality, voice input functionality, text input functionality, for interacting with and inputting data.

FIGS. 8A to 8F depict user interfaces 810A, 810B, 810C, 810D, 810E, 810F provided on a patient computing device to output various aspects of closed-loop programming recommendations. Specifically, these screens depict options of how a recommendation may be delivered, as part of a full recommendation and feedback campaign. In various examples, a patient's phone, computer, tablet, or programming remote control (or, multiple of these devices) may provide the illustrated user interfaces. The user interfaces 810A, 810B, 810C, 810D, 810E, 810F may provide an implementation of the graphical user interface 543 and the patient programming functionality discussed above. Additional menus and user interface functionality are not shown for purposes of simplicity.

FIG. 8A depicts the user interface 810A as including a graphical representation of a programming recommendation 811, a list of tasks 812, and program usage information 813. As will be understood, the delivery of a programming recommendation 811 may be coordinated with the use of the list of tasks and/or program usage information 813. FIG. 8B depicts the user interface 810B as including another screen to collect information regarding the programming recommendation, such as a feedback input screen 821 to collect information on whether the patient user tried or will try the recommendation. FIG. 8C depicts the user interface 810C as including another screen to collect information regarding the programming recommendation, such as a feedback input screen 822 to collect freeform text from the patient user regarding the use of the programming recommendation. FIG. 8D depicts the user interface 810D as including another screen to collect information regarding the programming recommendation, such as a feedback selection input screen 823 which provides a survey question regarding the programming recommendation. Finally, FIG. 8E depicts the user interface 810E as including an alternate screen to present informative content 824 regarding the feedback or the programming recommendation. FIG. 8F also depicts the user interface 810F as including an alternate screen to collect information regarding the recommendation, such as a feedback selection input screen 825 to collect freeform text from the patient if the user did not try the programming recommendation.

FIGS. 9A to 9D depict user interfaces 910A, 910B, 910C, 910D, provided on a patient computing device, for implementation of closed-loop programming recommendations. The user interfaces 910A, 910B, 910C, 910D may provide another implementation of the graphical user interface 543 and the patient programming functionality discussed above. Additional menus and user interface functionality are not shown for purposes of simplicity.

FIG. 9A depicts the user interface 910A as including a graphical representation of a list of tasks 921, including the presentation of a programming recommendation as one of the tasks. FIG. 9B depicts the user interface 910B as including a graphical representation of a survey question 922, including a selectable option to delay the use of the recommendation. FIG. 9C depicts the user interface 910C as including a graphical representation of instructions 923 to implement the recommendation, including a detailed list of program settings and guidance to the patient. FIG. 9D depicts the user interface 910D as including a graphical representation of additional instructions 924 (e.g., a tip) on how to implement the recommendation, such as information on the amount of usage of a particular program.

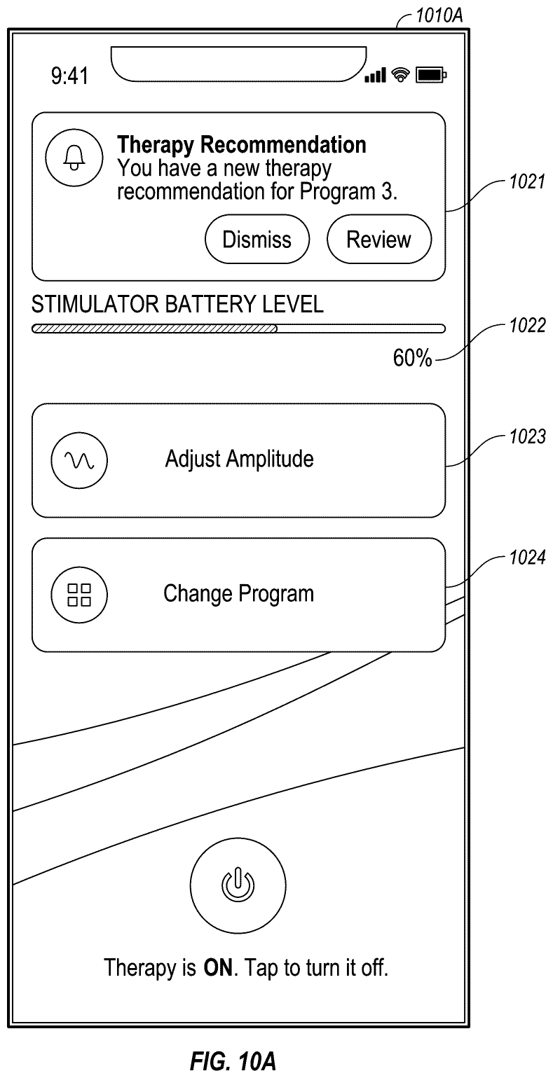

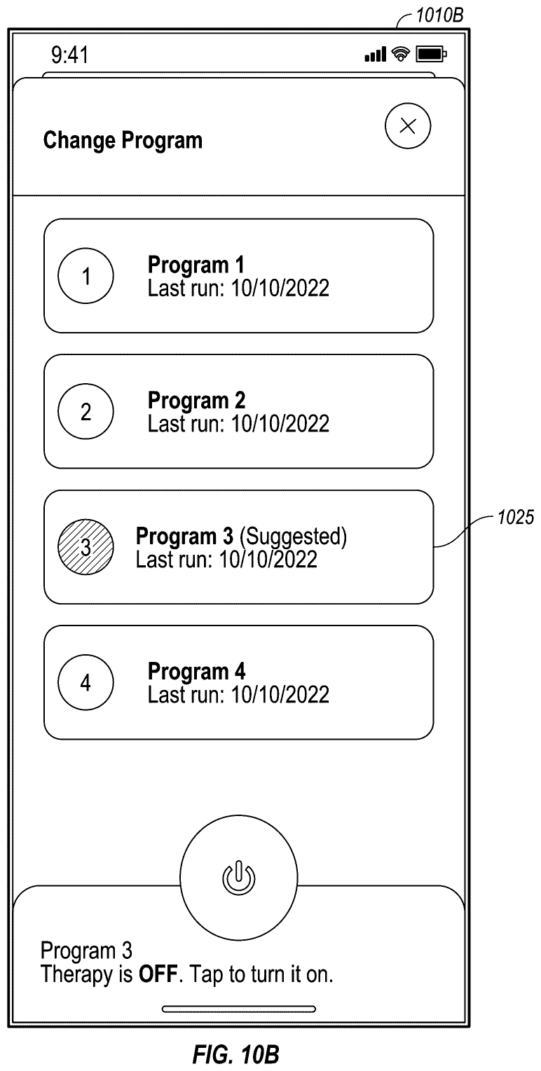

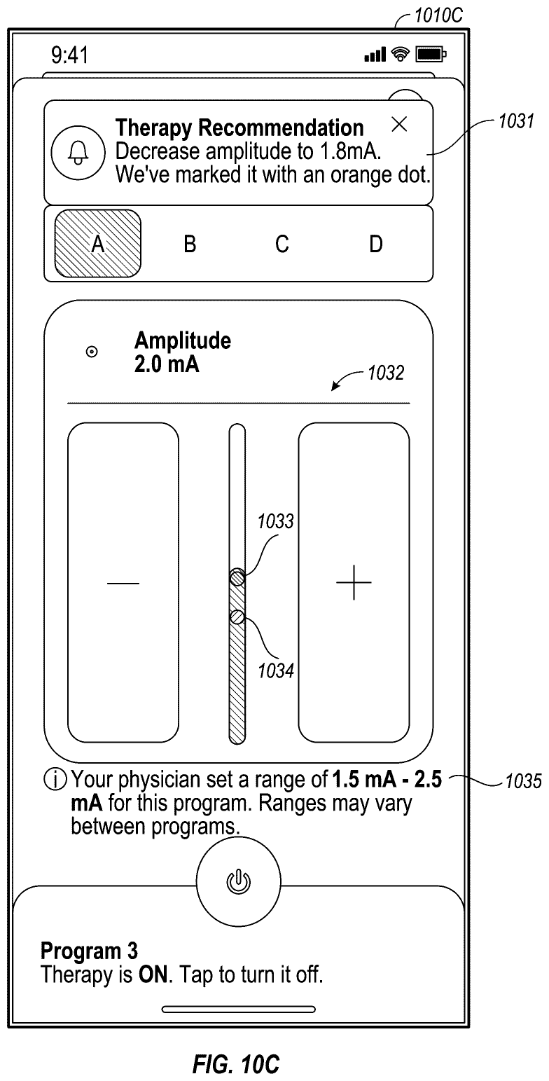

FIGS. 10A, 10B, and 10C depict user interfaces 1010A, 1010B, 1010C of a programming device (e.g., patient programming device 530) that is configured to receive and implement closed-loop programming recommendations. The user interfaces 1010A, 1010B, 1010C may provide an implementation of the user interface 531 and the programming implementation functionality discussed above. Additional menus and user interface functionality are not shown for purposes of simplicity.

FIG. 10A depicts the user interface 1010A as including a graphical representation of a programming recommendation. Specifically, this screen depicts a how the patient remote control may provide a “therapy recommendation” with a suggested program notification 1021 in a control screen of the remote control application. The recommendation may be delivered via the Internet to the patient's connected remote control application, which causes the remote control application to notify a user that a new recommendation is available.

FIG. 10B depicts the user interface 1010B as including a graphical representation of programs. Specifically, this screen depicts a recommended program 1025 (i.e., a suggested program) in a graphical listing of multiple programs. The recommended program 1025 may include one or more icons, text, color, or shading (e.g., a colored icon) to identify the recommended program.

FIG. 10C depicts the user interface 1010C as including a graphical representation of program settings. Specifically, this screen depicts a recommended adjustment notification 1031 to suggest that the user make a manual adjustment to an amplitude programming setting (adjustable via setting control 1032). The setting control 1032 includes an indicator on a graphical bar that shows a current setting 1033 and a recommended setting 1034 in a range of available values for neurostimulation waveform amplitude. Additional information 1035 may be presented regarding the type or amount of the manual adjustment. Here, the patient remains in control of whether to use the recommended value, and the amount of stimulation to apply based on the recommended value.

Although FIGS. 8A-9D and FIG. 10A-10C depict user interfaces from different types of devices, a single patient device with the same or similar user interface may be used to provide the functions of the patient programming device 530 and the patient computing device 520. For example, a smartphone may be capable of directly communicating with the stimulation device 221 and also operating a smartphone app to receive recommendations and program settings. Also, although some of the preceding examples refer to the use of individual programs, it will be understood that recommendations may also provide individual settings (e.g., changes in amplitude, intensity, waveforms, etc.) that can be implemented among multiple programs. Recommendations that change or affect device actions (e.g., program changes, programming setting changes, activities, etc.) may be delivered in other examples via one or more schedules, tasks, and goals.

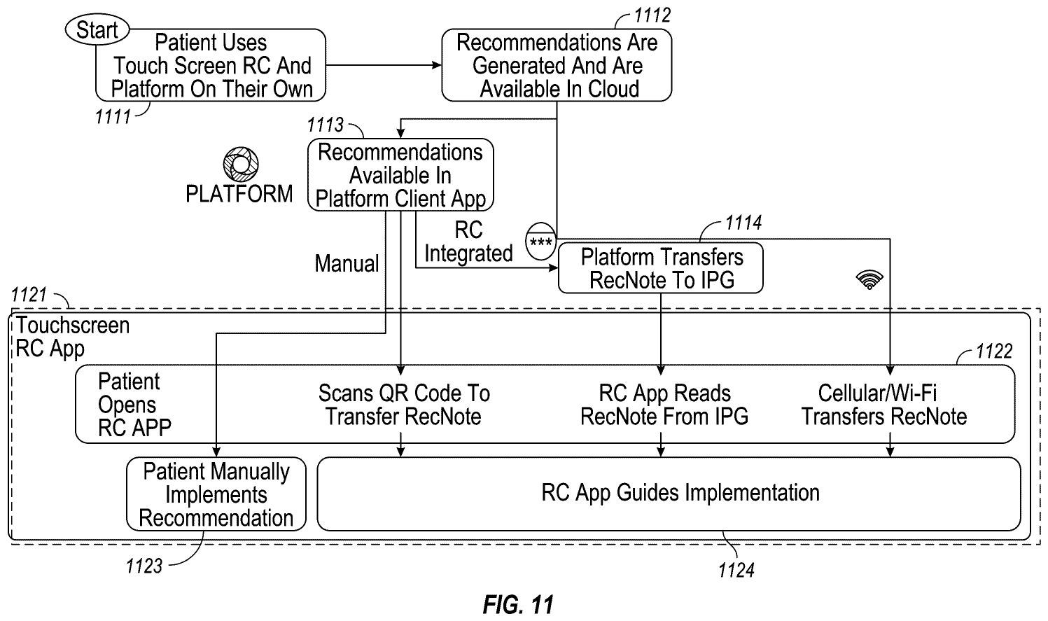

FIG. 11 illustrates data flows for implementation of closed-loop programming recommendations, based on the various examples discussed herein. The data flow begins with the patient use of the patient programming device 530 (e.g., a touch screen remote controller (RC)) and the patient computing device 520 (e.g., a smartphone app or website connected to a cloud service platform). Based on this use, programming recommendations are generated at operation 1112 via the cloud service platform, and made available to patient devices.

At operation 1113, recommendations are made available in a platform client app that is usable by a patient, such as a smartphone app on patient computing device 520. These available recommendations may be manually or automatically communicated to a touchscreen remote control app 1121 of a patient programming device 530, based on manual or automated actions. An example manual action to implement the recommendation is shown at operation 1123, where a patient opens and operates the remote control app 1121, and manually implements the recommendation by changing one or more programming settings of an existing or new program.

Automated or partially-automated actions are shown at operation 1124, where the remote control app 1121 guides the implementation of the recommendation. In a first example, the recommendation may be communicated to the remote control app 1121 by having the remote control app scan a QR code that embeds programming settings for the recommendation. In a second example, the cloud service platform transfers a recommendation note to the implantable pulse generator (e.g., stimulation device 221) at operation 1114, and the remote control app 1121 is used to enable or activate the implementation of the recommendation. In a third example, the recommendation is communicated to the remote control app 1121 (e.g., via a Wi-Fi network connection), and the remote control app 1121 is used to enable or activate the implementation of the recommendation.

A variety of patient behavior-adaptive methods may be incorporated for the delivery and content of recommendations, to enable a robust system that adapts recommendations to a patient's behavior and interactions with the overall neurostimulation treatment. For instance, consider the following examples that provide for customized recommendations. If a patient is viewing sleep scores in the app, entering more sleep score information, or reading helpful articles about sleep, then the system will target recommendations involving sleep. If a patient has a regular habit of checking his or her smartphone app and programming recommendations near a certain time (e.g., within 15 minutes of 5 pm), then the system may send recommendations shortly before this time to help the patient develop a habit. Other delivery examples may include: sending a recommendation when the patient looks at their phone and opens the smartphone app; sending a recommendation at a time when the patient historically changes programs; sending a recommendation when the system infers that the patient is nearby or handling the programming remote control; sending recommendations when a patient device is charging.

A variety of interactions can be accomplished with recommendations in the patient smartphone app. The interactions may include discrete actions such as dismiss, defer, delay, etc. The interactions may also capture feedback about the type or delivery of the recommendation (e.g., deliver more recommendations like this, fewer of this type of recommendations, deliver these type of recommendations at a later time, etc.). The patient interactions and feedback on the recommendations may be recorded to generate an ongoing history of past recommendations, including favorable ratings such as “likes” and shared likes. The patient interactions and feedback on the recommendations may also include directing feedback to a certain user group (e.g., the patient's clinical care team, a medical device company representative, a specific physician, or a caregiver). The user interface may also provide additional instructions or suggestions, such as a suggested progression of how to provide feedback and complete a recommendation (e.g., with discrete actions such as: View, Act, Feedback, Share). Other features of the patient smartphone app may include: a “For you” area to receive personalized messages and recommendations; a ‘My Messages’ or recommendation history area; information regarding the validity or source of the recommendation (whether provided as a personalized recommendation, or from another patient, care giver, rep, customer care, physician, algorithm, etc. . . . ); and a comparison of the recommendation (e.g., “People like you . . . ).

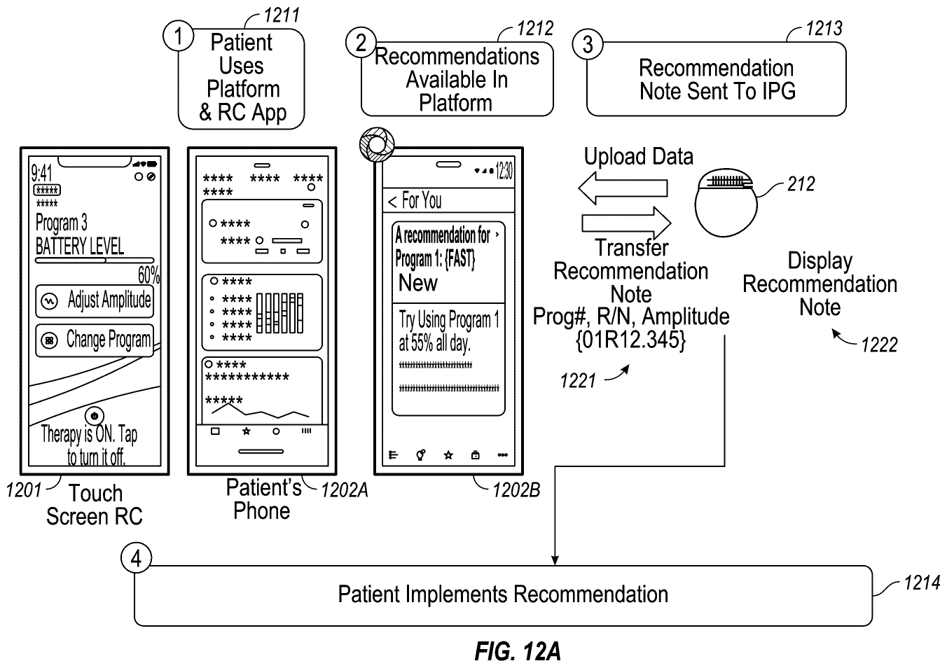

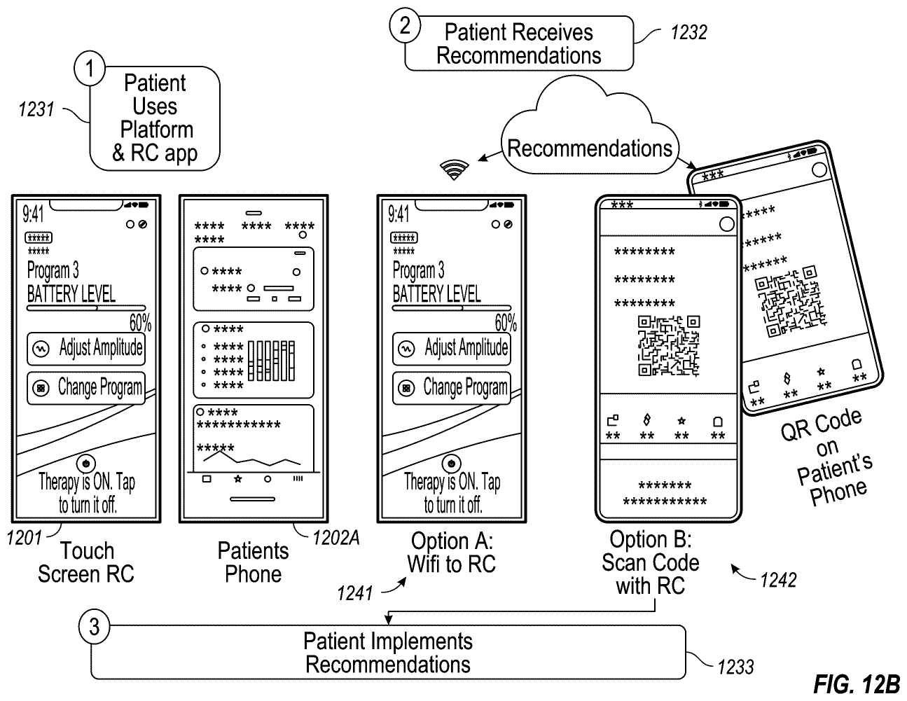

FIGS. 12A and 12B illustrate further workflows for patient implementation of closed-loop programming recommendations, according to an example. In FIG. 12A, a workflow includes a patient use of a platform (e.g., cloud-based platform) at operation 1211 that generates recommendations using a closed-loop programming model. The platform provides data to a user interface 1202A of the patient computing device 520 (e.g., patient smartphone) and a user interface 1201 of the patient programming device 530 (e.g., patient remote controller).

At operation 1211, various recommendations from the platform are made available at operation 1212 and surfaced to the patient in the user interface 1202B of the patient computing device 520. These recommendations are transferred to the patient programming device 530 at operation 1213 via a recommendation node 1221, and surfaced to the patient in the user interface 1201 to display at 1222. For example, the data may include information such as a program number, type of programming change, programming setting value (e.g., amplitude value), and the like. Other user interface screens, messages, and instructions may be used to guide the patient to implement the recommendation. At operation 1214, the patient provides a command in the patient computing device 520 or patient programming device 530 to implement the recommendation.

In further examples, the patient programming device 530 can receive data even if disconnected from the cloud platform or even if incapable of a network connection. This scenario is shown with the workflow provided in FIG. 12B. At operation 1231, similar to above, the patient uses the platform and the remote control application (e.g., the user interface 1201). However, at operation 1232, recommendations may be delivered to the remote control via one of two methods. With method 1241, a Wi-Fi or other wireless data connection is established between the patient computing device 520 and patient programming device 530 to transfer the recommendation node. With method 1242, a QR code that embeds or encodes the recommendation node (and programming values) is presented via the patient computing device 520, which can be read by a scanner or camera of the patient programming device 530. After either method of data transfer, at operation 1233, patient provides a command in the patient programming device 530 to implement the recommendation.

FIG. 13 illustrates, by way of example, an embodiment of a data processing flow for implementing recommended neurostimulation treatments in a human patient, which may integrate the closed-loop programming recommendation operations discussed above. Specifically, this data processing flow shows how a neurostimulation control system 1310 may perform patient state data processing 1314 and device state data processing 1316 functions to generate recommendation data 1312 (e.g., data providing recommended programming settings, as discussed above). The recommendation data 1312 can be generated based on programming data 1322 produced by a trained programming model 1320. The user adoption of the recommendations, in turn, will result in updated neurostimulation programming information 1342 that modifies the operation of the stimulation device 221.

In this example, the user interface 1300 (e.g., operating at the patient computing device 520) presents output data 1302 (e.g., recommendations, instructions, user guidance) including specific information relating to new and updated programs for the patient's stimulation device 221. The user interface 1300 also collects input data 1304 (e.g., survey answers, freeform text, other user interactions), as discussed above, which can be used for feedback on the programming recommendations, or which can modify the type and manner of programming. Such operations and functionality may be consistent with the user interfaces discussed above, although other data inputs and outputs may be used.

FIG. 13 also depicts the evaluation of device data 1330, such as sensor data 1332, therapy status data 1334, and other treatment aspects which may be obtained or derived from the stimulation device 221 or related neurostimulation programming and device operation. The device data 1330 can be further evaluated with patient state data processing 1314 and device state data processing 1316, to produce patient-specific programming recommendations. The inputs received in the user interface 1300 can be used to refine or customize the particular recommendations that are generated, including to change the type or characteristics (e.g., timing) of delivery of the recommendations. The trained programming model 1320 then generates new or updated programming data 1322 (e.g., programs, program parameter values) based on evaluation of the patient state data processing 1314, device state data processing 1316, similar patient or clinician data.

The remainder of the data processing flow illustrates how the neurostimulation control system 1310 implements programming, such as in a closed-loop (or partially-closed-loop) system that is driven by patient recommendations. A programming system 1340 uses programing information 1342 provided from the neurostimulation control system 1310 as an input to program implementation logic 1350. The program implementation logic 1350 may be implemented by a parameter adjustment algorithm 1354, which affects a neurostimulation program selection 1352 or a neurostimulation program modification 1356 (e.g., with such a program selection or modification directly caused by a recommendation). For instance, some parameter changes may be implemented by a simple modification to a program operation; other parameter changes may require a new program to be deployed. The results of the parameter or program changes or selection provides various stimulation parameters 1370 to the stimulation device 221, causing a different or new stimulation treatment effect 1360.

By way of example, operational parameters of the stimulation device which may be generated, identified, or evaluated by the neurostimulation control system 1310 may include amplitude, frequency, duration, pulse width, pulse type, patterns of neurostimulation pulses, waveforms in the patterns of pulses, and like settings with respect to the intensity, type, and location of neurostimulator output on individual or a plurality of respective leads. The neurostimulator may use current or voltage sources to provide the neurostimulator output, and apply any number of control techniques to modify the electrical simulation applied to anatomical sites or systems related to pain or analgesic effect. In various embodiments, a neurostimulator program may be defined or updated to indicate parameters that define spatial, temporal, and informational characteristics for the delivery of modulated energy, including the definitions or parameters of pulses of modulated energy, waveforms of pulses, pulse blocks each including a burst of pulses, pulse trains each including a sequence of pulse blocks, train groups each including a sequence of pulse trains, and programs of such definitions or parameters, each including one or more train groups scheduled for delivery. Characteristics of the waveform that are defined in the program may include, but are not limited to the following: amplitude, pulse width, frequency, total charge injected per unit time, cycling (e.g., on/off time), pulse shape, number of phases, phase order, interphase time, charge balance, ramping, as well as spatial variance (e.g., electrode configuration changes over time). It will be understood that based on the many characteristics of the waveform itself, a program may have many parameter setting combinations that would be potentially available for use.

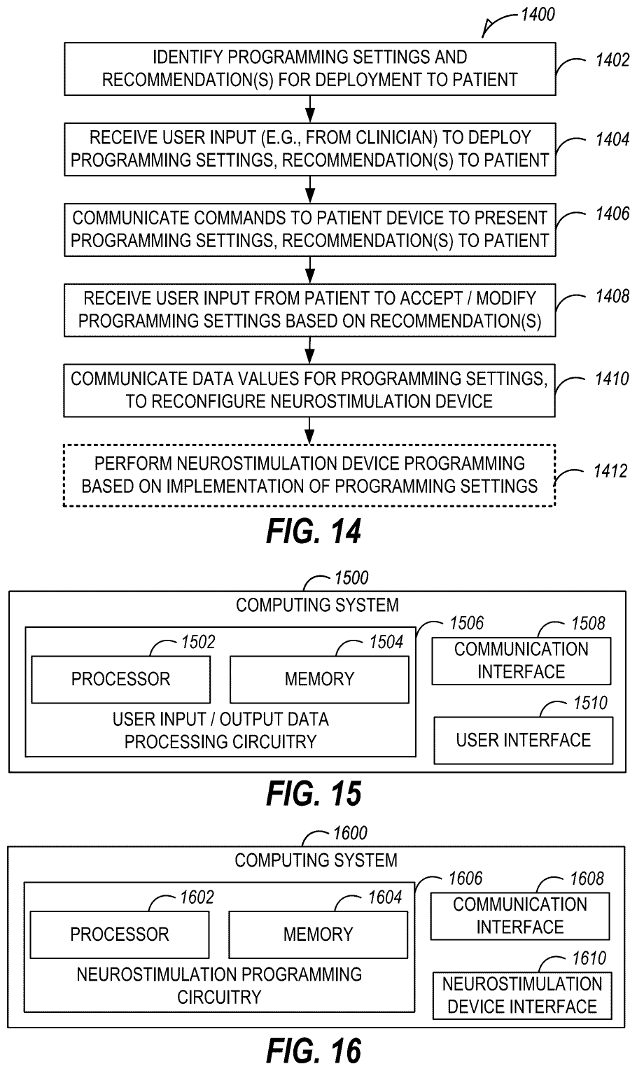

FIG. 14 illustrates, by way of example, an embodiment of a method 1400 implemented by a system or device configured to generate recommendations for neurostimulation programming, e.g., in connection with closed-loop programming operations for a neurostimulation treatment of a patient. For example, the method 1400 can be embodied by electronic operations performed by one or more computing systems or devices (including those at a network-accessible remote service) that are specially programmed to implement data analysis and/or neurostimulation data processing operations. In specific examples, the operations of the method 1400 may be implemented through the systems and data flows depicted above, at a single entity or at multiple locations.

In an example, the method 1400 begins at 1402 by identifying programming setting(s) and recommendation(s) for deployment to a patient. These settings and recommendations may be identified using a recommendation model and AI processing components such as a closed-loop or partially-closed-loop programming model as discussed above, although other types of algorithms, models, or rule-based systems may be used.

The method 1400 continues at 1404 by receiving user input from another user such as a medical user (e.g., clinician) or other authorized user (e.g., caregiver) associated with care of the patient to deploy the programming settings and/or recommendation(s) to a patient. In an example, one or more programming settings and recommendations that are identified using the recommendation model are communicated to a medical user device that includes a medical user interface, such as the clinician dashboard discussed above. This medical user interface enables the medical user to customize and activate the recommendation, and to provide a command to activate or change the recommendation (e.g., to use a particular programming setting, to change areas or types of treatments, to deploy particular schedules, etc.) as discussed above.

Consistent with the examples above, the medical user interface may present the recommendation in a prioritized listing of multiple available recommendations and outputs for the patient, and the medical user interface may receive various types of user input from the medical user that enable the customization and the activation of the recommendation. Here, customization may also include the ability to prioritize or to suggest a prioritization of a particular recommendation in a list of multiple available recommendations. Customization may also include user input received from the medical user to modify at least a part of the programming setting, or a presentation characteristic (e.g., timing, schedule, priority) associated with the recommendation. Additionally, in further examples, the system may ensure the safety of the user input (customization or activation) before deploying the recommendation to the patient and the patient neurostimulation system. For instance, a clinician medical user may be notified of possible operational conflicts or safety issues with use of the recommended settings.

The method 1400 continues at 1406 by communicating one or more commands to a patient device (e.g., patient smartphone, remote controller) to present the customized programming settings and recommendation(s) to a patient. The recommendation to use the programming setting may be associated with a medical user who recommends or specifies a time of delivery or validity (e.g., to configure a recommendation to be delivered or presented to the patient at a specific recommended time, and to be valid or usable only from date X to date Y, or to expire after time Z, etc.). Consistent with the examples above, the patient device may be a smartphone operable by the patient that presents a patient user interface in a software app installed on the smartphone. The patient device may also include a patient programming device or a hybrid device with programming and non-programming capabilities. In an example, the operations to communicate the commands and associated data values to the patient device occur after customization and activation of the recommendation by the medical user in the medical dashboard and medical user device.

The method 1400 continues at 1408 by receiving user input from a patient to accept or modify programming settings based on the recommendation(s). The patient device also may be used to receive a user command to manually input, change, activate, or control the data values, which reconfigures a programming setting (or, a program, or the use of a program) neurostimulation device.

The method 1400 continues at 1410 by communicating data values for programming settings, to reconfigure the neurostimulation device for treatment. In some examples, the patient device includes programming functionality to directly communicate the programming setting to the neurostimulation device. In other examples, the patient user interface is used to present encoded data values that are associated with the programming setting, and these encoded data values are readable by a patient programming device to then cause the patient programming device to reconfigure operation of the neurostimulation device according to the programming setting. In a specific example, such encoded data values are provided by a QR code, and are read by a camera of a patient programming device, as discussed above. In other examples, data may be encoded via other visual or sound outputs, such as in a patient user interface that provides encoded data with sound waves, which is captured and interpreted (decoded) by the patient programming device.

In further examples, the method 1400 continues at 1412 by optionally performing neurostimulation device programming based on implementation of the programming settings. Such programming may occur in connection with the use of a program, and the additional collection and evaluation of device data during use of the program that performs the neurostimulation treatment. As non-limiting examples, the data values that reconfigure the neurostimulation device may cause a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.

Other aspects of closed-loop programming and feedback may be provided within or subsequent to the method 1400. For example, feedback relating to the recommendation may be received in the patient device and processed, to generate subsequent recommendations that reconfigure the neurostimulation device to be customized to the patient based on the feedback. Further, the recommendation model may evaluate data values based on the feedback relating to the recommendation, based on feedback provided for one or more of: sleep, pain, movement, emotional state, medication state, mobility, or activity, in connection with use of the neurostimulation treatment. Such feedback can be obtained from the patient using one or more questionnaires, surveys, freeform text inputs, voice inputs, or sensor data observations, although other forms of data may be evaluated.

FIG. 15 illustrates, by way of example, a block diagram of an embodiment of a system 1500 (e.g., a computing system) for performing analysis of patient data, and generating or delivering recommendations in connection with the closed-loop neurostimulation data processing operations discussed above. The system 1500 may be integrated with or coupled to a computing device, a remote control device, patient programmer device, clinician programmer device, program modeling system, or other external device, deployed with neurostimulation treatment. In some examples, the system 1500 may be a networked device (server) connected via a network (or combination of networks) which communicates to one or more devices (clients) using a communication interface 1508 (e.g., communication hardware which implements software network interfaces and services). The network may include local, short-range, or long-range networks, such as Bluetooth, cellular, IEEE 802.11 (Wi-Fi), or other wired or wireless networks.

The system 1500 includes a processor 1502 and a memory 1504, which can be optionally included as part of user input/output data processing circuitry 1506. The processor 1502 may be any single processor or group of processors that act cooperatively. The memory 1504 may be any type of memory, including volatile or non-volatile memory. The memory 1504 may include instructions, which when executed by the processor 1502, cause the processor 1502 to implement data processing, or to enable other features of the user input/output data processing circuitry 1506. Thus, electronic operations in the system 1500 may be performed by the processor 1502 or the circuitry 1506.

For example, the processor 1502 or circuitry 1506 may implement any of the features of the method 1400 (such as operations 1402-1412) to obtain and process patient data, to generate recommendations, to allow modification and customization of recommendations, and to provide programming settings with delivered recommendations. It will be understood that the processor 1502 or circuitry 1506 may also implement aspects of the logic and processing described above, for use in various forms of closed-loop and partially-closed-loop device programming or related user interaction activities.

FIG. 16 illustrates, by way of example, a block diagram of an embodiment of a system 1600 (e.g., a computing system) implementing neurostimulation programming circuitry 1606 to cause programming of an implantable electrical neurostimulation device, for accomplishing the therapy objectives in a human subject based on the closed-loop recommendations discussed herein. The system 1600 may be operated by a clinician, a patient, a caregiver, a medical facility, a research institution, a medical device manufacturer or distributor, and embodied in a number of different computing platforms. The system 1600 may be a remote control device, patient programmer device, program modeling system, or other external device, including a regulated device used to directly implement programming commands and modification with a neurostimulation device. In some examples, the system 1600 may be a networked device connected via a network (or combination of networks) to a computing system operating a user interface computing system using a communication interface 1608. The network may include local, short-range, or long-range networks, such as Bluetooth, cellular, IEEE 802.11 (Wi-Fi), or other wired or wireless networks.

The system 1600 includes a processor 1602 and a memory 1604, which can be optionally included as part of neurostimulation programming circuitry 1606. The processor 1602 may be any single processor or group of processors that act cooperatively. The memory 1604 may be any type of memory, including volatile or non-volatile memory. The memory 1604 may include instructions, which when executed by the processor 1602, cause the processor 1602 to implement the features of the neurostimulation programming circuitry 1606. Thus, the electronic operations in the system 1600 may be performed by the processor 1602 or the circuitry 1606.

The processor 1602 or circuitry 1606 may directly or indirectly implement neurostimulation operations associated with the method 1400, including neurostimulation device programming based on data values delivered with modified or customized recommendations (operations 1410, 1412). The processor 1602 or circuitry 1606 may further provide data and commands to assist the processing and implementation of the programming using communication interface 1608 or a neurostimulation device interface 1610. It will be understood that the processor 1602 or circuitry 1606 may also implement other aspects of the device data processing or device programming functionality described above.

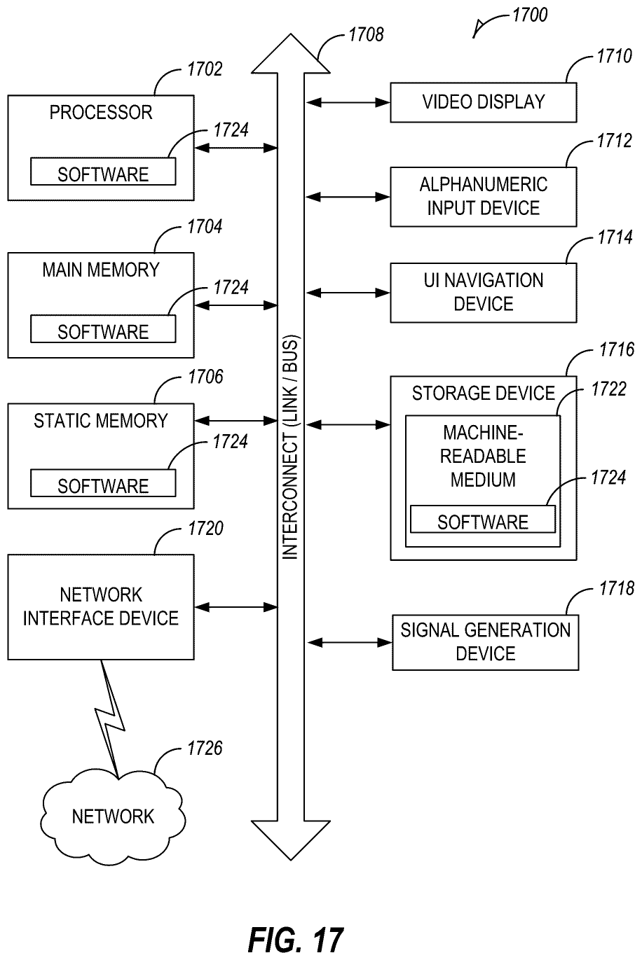

FIG. 17 is a block diagram illustrating a machine in the example form of a computer system 1700, within which a set or sequence of instructions may be executed to cause the machine to perform any one of the methodologies discussed herein, according to an example embodiment. In alternative embodiments, the machine operates as a standalone device or may be connected (e.g., networked) to other machines. In a networked deployment, the machine may operate in the capacity of either a server or a client machine in server-client network environments, or it may act as a peer machine in peer-to-peer (or distributed) network environments. The machine may be a personal computer (PC), a tablet PC, a hybrid tablet, a personal digital assistant (PDA), a mobile telephone, an implantable pulse generator (IPG), an external remote control (RC), a User's Programmer (CP), or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Similarly, the term “processor-based system” shall be taken to include any set of one or more machines that are controlled by or operated by a processor (e.g., a computer) to individually or jointly execute instructions to perform any one or more of the methodologies discussed herein.

Example computer system 1700 includes at least one processor 1702 (e.g., a central processing unit (CPU), a graphics processing unit (GPU) or both, processor cores, compute nodes, etc.), a main memory 1704 and a static memory 1706, which communicate with each other via a link 1708 (e.g., bus). The computer system 1700 may further include a video display unit 1710, an alphanumeric input device 1712 (e.g., a keyboard), and a user interface (UI) navigation device 1714 (e.g., a mouse). In one embodiment, the video display unit 1710, input device 1712 and UI navigation device 1714 are incorporated into a touch screen display. The computer system 1700 may additionally include a storage device 1716 (e.g., a drive unit), a signal generation device 1718 (e.g., a speaker), a network interface device 1720, and one or more sensors (not shown), such as a global positioning system (GPS) sensor, compass, accelerometer, or other sensor. It will be understood that other forms of machines or apparatuses (such as PIG, RC, CP devices, and the like) that are capable of implementing the methodologies discussed in this disclosure may not incorporate or utilize every component depicted in FIG. 17 (such as a GPU, video display unit, keyboard, etc.).

The storage device 1716 includes a machine-readable medium 1722 on which is stored one or more sets of data structures and instructions 1724 (e.g., software) embodying or utilized by any one or more of the methodologies or functions described herein. The instructions 1724 may also reside, completely or at least partially, within the main memory 1704, static memory 1706, and/or within the processor 1702 during execution thereof by the computer system 1700, with the main memory 1704, static memory 1706, and the processor 1702 also constituting machine-readable media.

While the machine-readable medium 1722 is illustrated in an example embodiment to be a single medium, the term “machine-readable medium” may include a single medium or multiple media (e.g., a centralized or distributed database, and/or associated caches and servers) that store the one or more instructions 1724. The term “machine-readable medium” shall also be taken to include any tangible (e.g., non-transitory) medium that is capable of storing, encoding or carrying instructions for execution by the machine and that cause the machine to perform any one or more of the methodologies of the present disclosure or that is capable of storing, encoding or carrying data structures utilized by or associated with such instructions. The term “machine-readable medium” shall accordingly be taken to include, but not be limited to, solid-state memories, and optical and magnetic media. Specific examples of machine-readable media include non-volatile memory, including but not limited to, by way of example, semiconductor memory devices (e.g., electrically programmable read-only memory (EPROM), electrically erasable programmable read-only memory (EEPROM)) and flash memory devices; magnetic disks such as internal hard disks and removable disks; magneto-optical disks; and CD-ROM and DVD-ROM disks.

The instructions 1724 may further be transmitted or received over a communications network 1726 using a transmission medium via the network interface device 1720 utilizing any one of a number of well-known transfer protocols (e.g., HTTP). Examples of communication networks include a local area network (LAN), a wide area network (WAN), the Internet, mobile telephone networks, plain old telephone (POTS) networks, and wireless data networks (e.g., Wi-Fi, 3G, and 4G LTE/LTE-A or 5G networks). The term “transmission medium” shall be taken to include any intangible medium that is capable of storing, encoding, or carrying instructions for execution by the machine, and includes digital or analog communications signals or other intangible medium to facilitate communication of such software.

The above detailed description is intended to be illustrative, and not restrictive. The scope of the disclosure should, therefore, be determined with references to the appended claims, along with the full scope of equivalents to which such claims are entitled.

What is claimed is:

1. A device to generate recommendations for neurostimulation device configuration, the device comprising:
one or more processors; and
one or more memory devices comprising instructions, which when executed by the one or more processors, cause the one or more processors to:
identify, with a recommendation model, a programming setting for use a neurostimulation device to control neurostimulation treatment of a patient;
communicate, to a patient device, a command to present a recommendation to use the programming setting, wherein the recommendation is customized by another user, and wherein the patient device includes a patient user interface to present the recommendation to the patient; and
communicate, to the patient device, data values associated with the programming setting, wherein the data values are used to reconfigure the neurostimulation device according to the programming setting.

2. The device of claim 1, wherein the another user is a medical user associated with care of the patient, and wherein the instructions further cause the one or more processors to:
communicate, to a medical user device, the programming setting identified with the recommendation model; and
receive, from the medical user device, a command to activate the recommendation for use of the programming setting, wherein the medical user device includes a medical user interface to enable the medical user to customize and activate the recommendation;
wherein operations to communicate the command and the data values to the patient device occur after customization and activation of the recommendation by the medical user.

3. The device of claim 1, wherein the patient user interface further includes encoded data values associated with the programming setting, and
wherein the encoded data values are readable by a patient programming device to cause the patient programming device to reconfigure operation of the neurostimulation device according to the programming setting.

4. The device of claim 1, wherein the patient device includes programming functionality to directly communicate the programming setting to the neurostimulation device.

5. The device of claim 1, wherein the patient device is a smartphone operable by the patient, and wherein the patient user interface is presented in a software app installed on the smartphone.

6. The device of claim 1, wherein the recommendation to use the programming setting is associated with a recommended time of delivery or validity for the patient device.

7. The device of claim 1, wherein the instructions further cause the one or more processors to:
receive, from the patient device, feedback relating to the recommendation, wherein subsequent recommendations to reconfigure the neurostimulation device are customized to the patient based on the feedback.

8. The device of claim 7, wherein the recommendation model is further configured to evaluate data values based on the feedback relating to the recommendation,
wherein the feedback relates to one or more of: sleep, pain, movement, emotional state, medication state, mobility, or activity, in connection with use of the neurostimulation treatment, and
wherein the feedback is obtained from the patient using one or more questionnaires, surveys, freeform text inputs, voice inputs, or sensor data observations.

9. The device of claim 1, wherein the recommendation model is further configured to evaluate device data collected during use of a program to perform the neurostimulation treatment.

10. The device of claim 1, wherein the patient device is further configured to receive a user command to manually input or control the data values, to reconfigure a program in the neurostimulation device according to the programming setting;
wherein the data values to reconfigure the neurostimulation device causes a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.

11. A method for generating recommendations for neurostimulation device configuration, comprising:
identifying, with a recommendation model, a programming setting for use a neurostimulation device to control neurostimulation treatment of a patient;
communicating, to a patient device, a command to present a recommendation to use the programming setting, wherein the recommendation is customized by another user, and wherein the patient device includes a patient user interface to present the recommendation to the patient; and
communicate, to the patient device, data values associated with the programming setting, wherein the data values are used to reconfigure the neurostimulation device according to the programming setting.

12. The method of claim 11, wherein the another user is a medical user associated with care of the patient, and wherein the method further comprises:
communicating, to a medical user device, the programming setting identified with the recommendation model; and
receiving, from the medical user device, a command to activate the recommendation for use of the programming setting, wherein the medical user device includes a medical user interface to enable the medical user to customize and activate the recommendation;
wherein communicating the command and the data values to the patient device occurs after customization and activation of the recommendation by the medical user.

13. The method of claim 11, wherein the patient user interface further includes encoded data values associated with the programming setting, and
wherein the encoded data values are readable by a patient programming device to cause the patient programming device to reconfigure operation of the neurostimulation device according to the programming setting.

14. The method of claim 11, wherein the patient device includes programming functionality to directly communicate the programming setting to the neurostimulation device.

15. The method of claim 11, wherein the patient device is a smartphone operable by the patient, and wherein the patient user interface is presented in a software app installed on the smartphone.

16. The method of claim 11, wherein the recommendation to use the programming setting is associated with a recommended time of delivery or validity for the patient device.

17. The method of claim 11, further comprising:
receiving, from the patient device, feedback relating to the recommendation, wherein subsequent recommendations to reconfigure the neurostimulation device are customized to the patient based on the feedback.

18. The method of claim 17, wherein the recommendation model is further configured to evaluate data values based on the feedback relating to the recommendation,
wherein the feedback relates to one or more of: sleep, pain, movement, emotional state, medication state, mobility, or activity, in connection with use of the neurostimulation treatment, and
wherein the feedback is obtained from the patient using one or more questionnaires, surveys, freeform text inputs, voice inputs, or sensor data observations.

19. The method of claim 11, wherein the recommendation model is further configured to evaluate device data collected during use of a program to perform the neurostimulation treatment.

20. The method of claim 11, wherein the patient device is further configured to receive a user command to manually input or control the data values, to reconfigure a program in the neurostimulation device according to the programming setting;
wherein the data values to reconfigure the neurostimulation device causes a change to one or more of: timing, amplitude, frequency, intensity, duration, pulse patterns, pulse shapes, a spatial location of pulses, waveform shapes, or a spatial location of waveform shapes, of modulated energy provided with a plurality of leads of the neurostimulation device.
